(19) United States
(12) Patent Application Publication
(10) Pub. No.: US 2022/0370605 A1
(43) Pub. Date: Nov. 24, 2022

(54) Inhibitor of DJ-1 for Use in Treating Immunoaging

(71) Applicants: Luxembourg Institute of Health (LIH), Luxembourg (LU); Université du Luxembourg, Esch-sur-Alzette (LU)

(72) Inventors: Feng He, Esch-sur-Alzette (LU); Markus Ollert, Mondorf-les-Bains (LU); Rudolf Balling, Belvaux (LU)

(73) Assignees: Luxembourg Institute of Health (LIH), Luxembourg (LU); Université du Luxembourg, Esch-sur-Alzette (LU)

(21) Appl. No.: 17/767,268

(22) PCT Filed: Oct. 8, 2020
(86) PCT No.: PCT/EP2020/078207

§ 371 Date: Apr. 7, 2022

(30) Foreign Application Priority Data
Oct. 8, 2019 (EP) 19201820.8
Dec. 10, 2019 (EP) 19214640.5

(51) Int. Cl. A61K 39/39; C12N 15/113

(52) U.S. Cl. CPC A61K 39/39; C12N 15/1137; A61K 2039/53

(57) ABSTRACT

The present invention relates to an inhibitor of DJ-1 (PARK7) for use in a method of treatment or prevention of immunoaging in a subject. In particular, the present invention relates to an inhibitor of DJ-1 for use in a method of treatment or prevention of an immunoaging-related disease in a subject, for use in a method of treatment or prevention of a premature aging disease in a subject, or for use in a method of treatment or prevention of vaccination inefficiency in a subject. In particular embodiments, the subject has been selected to have or has a premature aging disease, such as progeria, or the subject is an elderly subject.

### FIELD OF THE INVENTION

The invention relates to the medical field, in particular to the field of treatment of immunoaging. In particular, the present invention relates to treatment of premature aging diseases, immunoaging-related diseases, or vaccination inefficiency. The invention is particularly important to the elderly population, or to patients having a premature aging disease such as progeria.

### BACKGROUND OF THE INVENTION

Aging leads to a progressive functional decline of almost every organ in the body. It is one of the primary risk factors for the pathogenesis of various complex diseases. Given the increasing number of the elderly population worldwide, healthy aging is the key objective to maintain the stability and prosperity of the global community.

Immunoaging (also referred to as immunosenescence), denotes several changes, in particular age-related changes in the immune system that lead to the progressive decline of immunological competence, in particular in the elderly.

Among others, aging substantially alters T cell compositions and cellular phenotypes. In general, total naïve T cells (Tn; human, often identified as CD45RA⁺CCR7⁺CD62L⁺; mice, CD62L^{high}CD44^{low}) wane dramatically together with the increased effector memory (Tem; human, CD45RA⁻CCR7⁻CD62L⁻; mouse, CD62L^{low}CD44^{high}) and central memory T cells (Tcm; human, CD45RA⁻CCR7⁺CD62L⁺; mice, CD62L^{high}CD44^{high}). Since Tn are crucial for combating the novel and evolving pathogens, the dramatic decreased Tn pool represents defective adaptive immune responses towards new infections. Strikingly increased memory T cell compartment is mainly due to the latent infections over time, which means the T cell clones are dominated by recognizing some specific pervasive pathogens, therefore leading to the poor responsiveness to novel pathogens and diminished vaccination efficacy.

Besides the shift of naïve and memory compartments, there are some alterations of different T cell subsets with age. For instance, a subset of CD4 T cells, CD4⁺FOXP3⁺ T cells, known as Tregs that suppress responses of many types of effector immune cells were shown to be increased during aging. Another evident aging-associated feature of human T cells is the accumulation of the senescent CD27⁻CD28⁻ T cells. During the aging process, T cells first lose CD27 expression to become CD27⁻CD28⁺ intermediate differentiated T cells and next lose CD28 to generate CD27⁻CD28⁻ late-differentiated T cells.

Opposite to the downregulated expression of co-stimulatory markers, the expression of some inhibitory markers is strikingly upregulated in aged T cells. First, senescence associated markers like CD57, KLRG1 and CD85j, are dramatically upregulated in the elderly human CD8 T cells. Furthermore, the expression of some inhibitory molecules related to the T cell exhaustion are also increased in the aged T cells, such as PD-1, CTLA-4, TIM-3 and LAG-3, which might be triggered only by age in the absence of defined specific pathogens as demonstrated in mouse. Although exhaustion and senescence have different mechanisms and distinct transcriptional profiles, they are often intertwined to collectively contribute to the T cell aging phenotypes. Overall, increased exhausted and senescent T cells during aging together contribute to the poor T cell immune responsiveness.

Signs of an exhausted or senescent immune system have also been observed in patients suffering from a premature aging disease or a disease associated with premature aging. For example, it has been demonstrated that patients with the progeria Nijmegen breakage syndrome display many of the immune aging findings in circulating T cells (Meijers et al., Circulating T cells of patients with Nijmegen Breakage Syndrome show signs of senescence, J. Clin. Immunol., 2017, 37(2): 133-142). A similar observation has been made for patients with mutated ataxia-telangiectasia (Carney et al., Classical ataxia telangiectasia patients have a congenitally aged immune system with high expression of CD95, J Immunol., 2012, 189(1):261-8).

In an era of an ever-increasing elderly population worldwide, the immunoaging generates a huge social and economic burden, both in developed and developing countries. Accordingly, a need exists to develop further and improved substances or compositions for use in the treatment of immunoaging and immunoaging-related diseases or disorders in the elderly.

### SUMMARY OF THE INVENTION

The present inventors identified an unanticipated critical causative link between DJ-1 and immunoaging. In contrast to the natural aging process, wherein the frequency of Tregs increases while the frequency of naïve T cells decreases with age, the present inventors identified, by extensive experimental testing, a reduction in the Treg frequency of old DJ-1 knockout (KO) mice relative to that of the age- and gender-matched wildtype (WT) mice. Meanwhile, a significant increase of naïve T cells (Tn) while a significant decrease in the compartment of memory T cells was observed in old DJ-1 KO mice relative to that of WT mice. During the natural aging process, the exhaustion markers, such as PD-1, increase on T cells. On the contrary, this invention identified the loss of DJ-1 caused a reduction of the exhausted CD4 and CD8 T cells. Furthermore, to figure out whether the observed phenotypes are CD8 Tn-intrinsic or extrinsic, the inventors performed an adoptive transfer experiment of CD8 Tn into Rag1-deficient mice. Notably, the inventors observed a significantly decreased frequency of senescent CD8 memory T cells developed from CD8 Tn donor cells of old DJ-1 KO mice vs. WT mice in a lymphopenia-induced homeostatic proliferation. Interestingly, no effect of DJ-1 depletion was found in the young mice relative to the corresponding controls. These mice data demonstrated that DJ-1 depletion reduced immunoaging.

In line with mice data, the inventors have also observed a younger immune system when they analysed the blood from the patient with DJ-1 loss-of-function deficiency relative to the age- and gender-matched healthy siblings with DJ-1 heterozygous mutation. More specifically, in the patient with DJ-1 deficiency vs. the siblings, reduced exhaustion and senescence in T cells were observed accompanied with an enhanced TCR repertoire diversity, which is supposed to be reduced during natural aging. In short, the inventors have demonstrated that DJ-1 depletion plays an unexpected and pivotal role in slowing down immunoaging.

Accordingly, the inventors have realised the medical use of a DJ-1 inhibitor in a method of treatment or prevention of immunoaging in a subject. In addition, the inventors demonstrate the potential of DJ-1 inhibitor as a check point inhibitor.

In a first aspect, the invention provides an inhibitor of DJ-1 for use in treating or preventing immunoaging in a subject. Preferably, the subject is a human subject.

Certain embodiments provide:
the inhibitor of DJ-1 for use in rejuvenating the immune system in a subject, in particular in a subject having been selected (e.g. diagnosed) to have or having a premature aging disease, such as progeria, or in an elderly subject.
the inhibitor of DJ-1 for use in treating or preventing a premature aging disease, such as Hutchinson-Gilford progeria syndrome (HGPS), in a subject, in particular in a subject having been selected (e.g. diagnosed) to have or having a premature aging disease, such as progeria.
the inhibitor of DJ-1 for use in treating or preventing an immunoaging-related disease, such as cancer or an infectious disease, in a subject, wherein the subject has been selected (e.g. diagnosed) to have or has a premature aging disease, such as progeria, or wherein the subject is an elderly subject.

In addition, the present inventors found that an inhibitor of DJ-1 administered in conjunction with or as part of an immunogenic composition boosts the immune response of an elderly subject upon vaccination. Hence, the present inventors realised the medical use of a DJ-1 inhibitor in the treatment of vaccination inefficiency in a subject, in particular in a subject having been selected (e.g. diagnosed) to have or having a premature aging disease, such as progeria, or in an elderly subject.

An aspect thus provides the use of an inhibitor of DJ-1 as an adjuvant, in particular as an adjuvant in a cancer vaccine.

Further aspects provide:
A immunogenic composition comprising: an inhibitor of DJ-1 as defined herein, and a compound or composition capable of inducing an immune response. Preferably, the immunogenic composition is a cancer vaccine.
A kit of parts comprising: an inhibitor of DJ-1 as defined herein, and a compound or composition capable of inducing an immune response.

A further aspect of the invention relates to the use of an inhibitor of DJ-1 as defined herein as a checkpoint inhibitor. Indeed, as DJ-1 depletion can significantly decrease PD-1 expression among CD4 and CD8 T cells in old mice, DJ-1 inhibitors can be used as an immune checkpoint inhibitor. Meanwhile, DJ-1 inhibitors can be used to reduce Treg frequency and thus enhancing the response of effector T cells to fight against tumors. Immune checkpoint inhibitors are of particular interest in the treatment of diseases, such as but not limited to cancer, more particularly in patients which are identified to be likely to be susceptible to the treatment with a check point inhibitors.

An embodiment thus relates to the inhibitor of DJ-1 as defined herein, an immunogenic composition as defined herein, or a kit of parts as defined herein, for use in treating or preventing vaccination inefficiency in a subject; preferably wherein the subject has been selected (e.g. diagnosed) to have or has a premature aging disease or wherein the subject is an elderly subject.

The above and other characteristics, features and advantages of the present invention will become apparent from the following detailed description, which illustrate, by way of example, the principles of the invention.

### BRIEF DESCRIPTION OF DRAWINGS

FIG. 1 illustrate that DJ-1 depletion reduces Tregs in old mice. FIG. 1A represents a graph showing the percentage of FOXP3+ CD4 T cells in the spleen (Sp) and peripheral lymph nodes (LN) from DJ-1^{-/-} and DJ-1^{+/+} WT littermates. FIG. 1B illustrates total Tregs between old DJ-1^{-/-} and DJ-1^{+/+} controls around 45 weeks old. Data are mean±SD. FIG. 1C provides representative flow-cytometry plots of FOXP3 and CD4 expression on total CD4 T cells of old mice, DJ-1^{-/-} (left) or DJ-1^{+/+} (right). FIG. 1D represents a graph showing the percentages of CD8⁺ T cells in the spleen (Sp) and peripheral lymph nodes (LN) from DJ-1^{-/-} and DJ-1^{+/+} WT littermates. FIG. 1E represents a graph showing the relative ratios between CD8⁺ T cells and FOXP3⁺CD4⁺ Tregs in the spleen (Sp) and peripheral lymph nodes (LN) from DJ-1^{-/-} and DJ-1^{+/+} WT littermates. FIG. 1F represents a graph showing the expression of IL-10 in CD4 T cells from young or old mice after in-vitro stimulation using PMA/ionomycin for 5 h. The P-values are determined by a two-tailed Student's t-test. n.s.: not significant, *P<=0.05, **P<=0.01 and ***P<=0.001.

FIG. 2 illustrates that DJ-1 depletion halts immunoaging in old mice. FIG. 2A provides representative flow-cytometry plots of CD44 and CD62L expression on total CD4 T cells, DJ-1^{-/-} (left) or DJ-1^{+/+} (right). FIG. 2B provides representative flow-cytometry plots of CD44 and CD62L expression on total CD8 T cells, DJ-1^{-/-} (left) or DJ-1^{+/+} (right); Q1: effector and memory T-cells (Tem), Q2: central memory T-cells (Tcm); Q3: naive T-cells (Tn).

FIG. 3 represents graphs illustrating the percentages of CD44^{low} CD62L^{high} (left) and CD44^{high} CD62L^{low} (right) cells among total CD4 T cells in the spleen (Sp) and peripheral lymph nodes (LN) from DJ-1^{-/-} and DJ-1^{+/+} WT littermates. Results represent at least four independent experiments. Data are mean±SD. The P-values are determined by a two-tailed Student's t-test. ns, not significant, *P<=0.05, **P<=0.01 and ***P<=0.001.

FIG. 4 represents graphs illustrating the percentages of CD44^{low} CD62L^{high} (left) and CD44^{high} CD62L^{low} (right) cells among total CD8 T cells in the spleen (Sp) and peripheral lymph nodes (LN) from DJ-1^{-/-} and DJ-1^{+/+} WT littermates. Results represent at least four independent experiments. Data are mean±SD. The P-values are determined by a two-tailed Student's t-test. ns, not significant, *P<=0.05, **P<=0.01 and ***P<=0.001.

FIG. 5 represents representative histogram overlays of PD-1 expression among total CD4+ (spleen) (left) or CD8+ (spleen) (right) T cells of old mice. Grey: DJ-1^{-/-} KO mice; Black: DJ-1^{+/+} WT mice.

FIG. 6 represents graphs illustrating percentages of PD-1⁺ cells among total CD4 (left) or CD8 (right) T cells in the spleen (Sp) and peripheral lymph nodes (LN) from DJ-1^{-/-} and DJ-1^{+/+} WT littermates. Data are mean±SD. The P-values are determined by a two-tailed Student's t-test. ns, not significant, *P<=0.05, **P<=0.01 and ***P<=0.001.

FIG. 7 illustrates that DJ-1 deficiency enhances antigen-specific response in old but not young mice. EAE clinical scores on the indicated days following immunization with MOG₃₅₋₅₅. The number of age- and gender-matched mice per group are indicated in the figures. Data are representative for young mice and a summary of 2 independent experiments for old mice groups. Young mice (˜12 weeks, left) and old mice (˜45 weeks, right). Grey round dots: DJ-1^{-/-} KO mice; Black triangular dots: DJ-1^{+/+} WT mice. Results represent two independent experiments. Data are mean±SD. The P-values are determined by a two-tailed paired Student's t-test. ns, not significant, *P<=0.05, **P<=0.01 and ***P<=0.001.

FIG. 8 illustrates that DJ-1 intrinsically slows down senescence of CD8 T cells. FIG. 8A represents a schematic drawing of the adoptive transfer experiments. FIG. 8B, FIG. 8C illustrate representative KLRG1 expression plots among different CD8 T cell subsets (FIG. 8B, among total CD8 T cells; FIG. 8C, among CD8 Tem) developed from naïve CD8 T cells of old donor mice. FIG. 8D shows percentages of CD69+ cells among total CD8 T cells (left) or among CD8 Tem (right) from young or old, WT or DJ-1^{-/-} KO donor mice. The P-values are determined by a two-tailed Student's t-test. ns (or not marked), not significant, *P<=0.05, **P<=0.01 and ***P<=0.001.

FIG. 9 represents an extract of the pedigree of the family carrying the c.192G>C mutation in the DJ-1 gene indicating the three participants (P3, P1 and P2). The half black square/circle indicates male/female individuals heterozygous for the DJ-1 mutation, the black square indicates the patient carrying the homozygous DJ-1 mutation. *Obligate heterozygous mutation carrier although dead at the time of the initial study.

FIG. 10 illustrates that DJ-1 ablation confers PD patients a juvenile immune system.

FIG. 10 provides expression of CD45RO and CD28 (FIG. 10A), CD28 and CD27 (FIG. 10B), CD45RO and CD57 (FIG. 10C) on peripheral blood CD8 T cells. FIG. 10D illustrates expression of PD-1 and T-bet on CD8 T cells. FIG. 10E illustrates expression of CCR7 and CD45RO on CD4 T cells. The enlarged number in the corresponding gate represents the corresponding percentage out of the parent population. FIG. 10F represents histogram overlays of ICOS expression on CD4+FOXP3+, CD4 and CD8 T cells between P1 (heterozygous mutation), P2 (homozygous mutation) and P3 (heterozygous). The number represents geometric mean of the ICOS intensity of the given cell type.

FIG. 11 illustrates that DJ-1 deficiency downregulates exhaustion and senescence associated pathways in CD8 T cells. FIG. 11A represents a graph illustrating levels of mRNA expression of exhaustion genes, LAG3 and TIM3 of CD8 T cells from three participants' peripheral blood. FIG. 11B represents a graph illustrating mRNA expression of immunosenescence genes, CD57, CD85j and KLRG1, anti-apoptosis gene BCL2, costimulatory receptor genes, CD28 and CD27, of CD8 T cells in the peripheral blood. FIG. 11C represents a graph illustrating comparison of mRNA expression of NK cell-related genes, KIR3DX1, KLRD1 and KLRF1 in CD8 T cells. FIG. 11D represents a graph illustrating mRNA expression of cyclin-dependent kinase inhibitor genes of CD8 T cells from the peripheral blood. Light grey: P1 CD8 T cells (heterozygous mutation), dark grey: P2 CD8 T cells (homozygous mutation), and black: P3 CD8 T cells (heterozygous).

FIG. 12 illustrates that DJ-1 deficiency enhances human TCR beta repertoire. FIG. 12A represents a graph illustrating the lower bound of TCR repertoire of CD4 Tn (right) and CD8 Tn (left) by applying nonparametric statistics using the iChaol estimator (ImmunoSEQ Analyzer 3.0, Adaptive Biotechnologies). FIG. 12B represents a graph showings the richness of TCR repertoire of CD4 Tn (right) and CD8 Tn (left) by applying a nonparametric empirical Bayes estimation using the Efron Thisted estimator (extrapolation value is 120K from ImmunoSEQ Analyzer 3.0, Adaptive Biotechnologies). FIG. 12C represents a graph showing the clonality of TCR repertoire of CD4 Tn (right) and CD8 Tn (left) (using ImmunoSEQ Analyzer 3.0, Adaptive Biotechnologies). Clonality is equal to 1—normalized Shannon's Entropy.

FIG. 13 represents graphs illustrating the cytokine levels in the supernatant of splenocytes of FLUAD-vaccinated mice followed by the restimulation of different amount of FLUAD vaccine. FIG. 13a, FIG. 13 b, Interleukin 2 (IL2) levels in the supernatant of splenocytes isolated from young adult (FIG. 13 a) or aged (FIG. 13 b) DJ-1^{+/+} or DJ-1^{-/-} mice vaccinated with FLUAD followed by the restimulation of different amount of FLUAD vaccines (0, 1:50 or 1:100 diluted in the media) for 3 days. FIG. 13 c, d, Interferon gamma (“IFNg”) levels in the supernatant of splenocytes isolated from young adult (FIG. 13 c) or aged (FIG. 13 d) DJ-1^{+/+} or DJ-1^{-/-} mice vaccinated with FLUAD followed by the restimulation of different amount of FLUAD vaccines (0, 1:50 or 1:100 diluted in the media) for 3 days. In both 1:50 and 1:100 dilution in FIG. 13 d, individual mice with obvious higher responses were labelled with numbers. The change direction from aged DJ-1^{+/+} mice to aged DJ-1^{-/-} mice were marked with an arrow in both FIG. 13 b and FIG. 13 d. Each symbol represents one individual mouse.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any 3 or more, 4 or more, 5 or more, 6 or more, or 7 or more etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The present invention relates to an inhibitor of DJ-1 for use in treating or preventing immunoaging, a premature aging disease, an immunoaging-related disease, and/or vaccination inefficiency in a subject. By extensive experimental testing, the present inventors realised that DJ-1 deficiency decreases Treg genesis and development of memory T cells, halts T cell exhaustion and senescence and increases antigen-specific responses, thus attenuating immunoaging.

In a first aspect, the present invention relates to an inhibitor of DJ-1 (PARK7) for use in treating or preventing immunoaging in a subject.

The reference to “protein deglycase DJ-1” or “DJ-1” denotes the DJ-1 peptide, polypeptide, protein, or nucleic acid, as commonly known under said designation in the art. By means of additional guidance, DJ-1 is also known as DJ1, Parkinson disease protein 7 (PARK7), HEL-S-67p, Parkinsonism associated deglycase, GATD2. The terms denote DJ-1 nucleic acids, as well as DJ-1 peptides, polypeptides and proteins, as apparent from the context. The term “DJ-1 polypeptide” as used herein is synonymous with “DJ-1 protein”. The protein is encoded in humans by the PARK7 gene.

By means of an example, human DJ-1 mRNA is annotated under NCBI Genbank (http://www.ncbi.nlm.nih.gov/) accession numbers NM_007262.5 (“transcript variant 1”, SEQ ID NO: 1), NM_001123377.1 (“transcript variant 2”, SEQ ID NO: 2), or XM_005263424.3 (“transcript variant X1”).

The human DJ-1 mRNA sequence annotated
under NCBI Reference Sequence NM_007262.5
is reproduced below (SEQ ID NO: 1):
GCGTTCATTTTCAGCCTGGTGTGGGGTGAGTGGTACCCAACGGGCCGGGG
CGCCGCGTCCGCAGGAAGAGGCGCGGGGTGCAGGCTTGTAAACATATAAC
ATAAAAATGGCTTCCAAAAGAGCTCTGGTCATCCTGGCTAAAGGAGCAGA
GGAAATGGAGACGGTCATCCCTGTAGATGTCATGAGGCGAGCTGGGATTA
AGGTCACCGTTGCAGGCCTGGCTGGAAAAGACCCAGTACAGTGTAGCCGT
GATGTGGTCATTTGTCCTGATGCCAGCCTTGAAGATGCAAAAAAAGAGGG
ACCATATGATGTGGTGGTTCTACCAGGAGGTAATCTGGGCGCACAGAATT
TATCTGAGTCTGCTGCTGTGAAGGAGATACTGAAGGAGCAGGAAAACCGG
AAGGGCCTGATAGCCGCCATCTGTGCAGGTCCTACTGCTCTGTTGGCTCA
TGAAATAGGTTTTGGAAGTAAAGTTACAACACACCCTCTTGCTAAAGACA
AAATGATGAATGGAGGTCATTACACCTACTCTGAGAATCGTGTGGAAAAA
GACGGCCTGATTCTTACAAGCCGGGGGCCTGGGACCAGCTTCGAGTTTGC
GCTTGCAATTGTTGAAGCCCTGAATGGCAAGGAGGTGGCGGCTCAAGTGA
AGGCTCCACTTGTTCTTAAAGACTAGAGCAGCGAACTGCGACGATCACTT
AGAGAAACAGGCCGTTAGGAATCCATTCTCACTGTGTTCGCTCTAAACAA
AACAGTGGTAGGTTAATGTGTTCAGAAGTCGCTGTCCTTACTACTTTTGC
GGAAGTATGGAAGTCACAACTACACAGAGATTTCTCAGCCTACAAATTGT
GTCTATACATTTCTAAGCCTTGTTTGCAGAATAAACAGGGCATTTAGCAA
ACTACTGATTGTTTCTTGTTTTGTCTCTCATTTCTTTTGTGAAATTAAAT
TCCGTATCACCTTCATTTGCAGCTCTTAACTGTCCATATGGCACTGAAAT
AAAAGAACAGTGACCACATTTTACACAGCAAGGAGGAAAGGCATACAAAC
AGAATTTAAGAGGCTTGTGATTTTCTCTGCTTATTAGCTGTGTGTTTTTA
ATGTGCTATTAAAAAATACCAATGAGG
The human DJ-1 mRNA sequence annotated
under NCBI Reference Sequence NM_001123377.1
is reproduced below (SEQ ID NO: 2):
TGAGTCTGCGCAGTGTGGGGCTGAGGGAGGCCGGACGGCGCGCGTGCGTG
CTGGCGTGCGTTCATTTTCAGCCTGGTGTGGGGCTTGTAAACATATAACA
TAAAAATGGCTTCCAAAAGAGCTCTGGTCATCCTGGCTAAAGGAGCAGAG
GAAATGGAGACGGTCATCCCTGTAGATGTCATGAGGCGAGCTGGGATTAA
GGTCACCGTTGCAGGCCTGGCTGGAAAAGACCCAGTACAGTGTAGCCGTG
ATGTGGTCATTTGTCCTGATGCCAGCCTTGAAGATGCAAAAAAAGAGGGA
CCATATGATGTGGTGGTTCTACCAGGAGGTAATCTGGGCGCACAGAATTT
ATCTGAGTCTGCTGCTGTGAAGGAGATACTGAAGGAGCAGGAAAACCGGA
AGGGCCTGATAGCCGCCATCTGTGCAGGTCCTACTGCTCTGTTGGCTCAT
GAAATAGGTTTTGGAAGTAAAGTTACAACACACCCTCTTGCTAAAGACAA
AATGATGAATGGAGGTCATTACACCTACTCTGAGAATCGTGTGGAAAAAG
ACGGCCTGATTCTTACAAGCCGGGGGCCTGGGACCAGCTTCGAGTTTGCG
CTTGCAATTGTTGAAGCCCTGAATGGCAAGGAGGTGGCGGCTCAAGTGAA
GGCTCCACTTGTTCTTAAAGACTAGAGCAGCGAACTGCGACGATCACTTA
GAGAAACAGGCCGTTAGGAATCCATTCTCACTGTGTTCGCTCTAAACAAA
ACAGTGGTAGGTTAATGTGTTCAGAAGTCGCTGTCCTTACTACTTTTGCG
GAAGTATGGAAGTCACAACTACACAGAGATTTCTCAGCCTACAAATTGTG
TCTATACATTTCTAAGCCTTGTTTGCAGAATAAACAGGGCATTTAGCAAA
CTAAAAAAAAAAAAAAAAAAA
By means of an example, human DJ-1
protein sequence is annotated under
NCBI Genbank accession numbers NP_009193.2,
NP_001116849.1, and XP_005263481.1, and
Uniprot (www.uniprot.org) accession number
Q99497, and is further reproduced below
(SEQ ID NO: 3):
MASKRALVILAKGAEEMETVIPVDVMRRAGIKVTVAGLAGKDPVQCSRDV
VICPDASLEDAKKEGPYDVVVLPGGNLGAQNLSESAAVKEILKEQENRKG
LIAAICAGPTALLAHEIGFGSKVTTHPLAKDKMMNGGHYTYSENRVEKDG
LILTSRGPGTSFEFALAIVEALNGKEVAAQVKAPLVLKD

In certain embodiments, the amino acid sequence of said DJ-1 polypeptide is as set forth in GenBank accession no. NP_001116849.1.

By means of an example, human DJ-1 gene is annotated under NCBI Genbank Gene ID 11315.

By means of an example, mouse DJ-1 mRNA is annotated under NCBI Genbank (http://www.ncbi.nlm.nih.gov/) accession number NM_020569.3.

The amino acid sequence of the protein that encodes mouse (Mus musculus) DJ-1 can have or comprise Uniprot number Q99LX0-1 or is annotated under NCBI Genbank accession number NP_065594.2. The amino acid sequence of the protein that encodes DJ-1 in chicken (Gallus gallus) can have or comprise Uniprot number Q8UW59-1. The amino acid sequence of the protein that encodes DJ-1 in rat (Rattus norvegicus) can have or comprise Uniprot number 088767-1.

A skilled person can appreciate that any sequences represented in sequence databases or in the present specification may be of precursors of peptides, polypeptides, proteins, or nucleic acids and may include parts which are processed away from mature molecules.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

Without limitation, protein, polypeptides or peptides can be produced recombinantly by a suitable host or host cell expression system and isolated therefrom (e.g., a suitable bacterial, yeast, fungal, plant or animal host or host cell expression system), or produced recombinantly by cell-free transcription and/or translation, or non-biological protein, polypeptide or peptide synthesis.

The term “nucleic acid” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Exemplary modified nucleobases include without limitation 5-substituted pyrimidines, 6-azapyrimidines and N-2, N-6 and 0-6 substituted purines, including 2-aminopropyladenine, 5-propynyluracil and 5-propynylcytosine. In particular, 5-methylcytosine substitutions have been shown to increase nucleic acid duplex stability and may be preferred base substitutions in for example antisense agents, even more particularly when combined with 2′-O-methoxyethyl sugar modifications. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleic acid molecules comprising at least one ribonucleoside unit may be typically referred to as ribonucleic acids or RNA. Such ribonucleoside unit(s) comprise a 2′-OH moiety, wherein —H may be substituted as known in the art for ribonucleosides (e.g., by a methyl, ethyl, alkyl, or alkyloxyalkyl). Preferably, ribonucleic acids or RNA may be composed primarily of ribonucleoside units, for example, ≥80%, ≥85%, ≥90%, ≥95%, ≥96%, ≥97%, ≥98%, ≥99% or even 100% (by number) of nucleoside units constituting the nucleic acid molecule may be ribonucleoside units. Nucleic acid molecules comprising at least one deoxyribonucleoside unit may be typically referred to as deoxyribonucleic acids or DNA. Such deoxyribonucleoside unit(s) comprise 2′-H. Preferably, deoxyribonucleic acids or DNA may be composed primarily of deoxyribonucleoside units, for example, ≥80%, ≥85%, ≥90%, ≥95%, ≥96%, ≥97%, ≥98%, ≥99% or even 100% (by number) of nucleoside units constituting the nucleic acid molecule may be deoxyribonucleoside units. Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof.

The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, can be recombinant, i.e., produced by recombinant DNA technology, and/or can be, partly or entirely, chemically or biochemically synthesised. Without limitation, nucleic acids can be produced recombinantly by a suitable host or host cell expression system and isolated therefrom (e.g., a suitable bacterial, yeast, fungal, plant or animal host or host cell expression system), or produced recombinantly by cell-free transcription, or non-biological nucleic acid synthesis. A nucleic acid can be double-stranded, partly double stranded, or single-stranded. Where single-stranded, the nucleic acid can be the sense strand or the antisense strand. In addition, nucleic acid can be circular or linear.

The reference to any peptide, polypeptide, protein, or nucleic acid, corresponds to the peptide, polypeptide, protein, or nucleic acid, commonly known under the respective designations in the art. The terms encompass such peptides, polypeptides, proteins, or nucleic acids, of any organism where found, and particularly of animals, preferably warm-blooded animals, more preferably vertebrates, yet more preferably mammals, including humans and non-human mammals, still more preferably of humans.

In certain embodiments, the DJ-1 peptide, polypeptide, protein, or nucleic acid is of animal origin, preferably warm-blooded animal origin, more preferably vertebrate origin, yet more preferably mammalian origin, including human origin and non-human mammalian origin, still more preferably human origin.

The terms particularly encompass such peptides, polypeptides, proteins, or nucleic acids, with a native sequence, i.e., ones of which the primary sequence is the same as that of the peptides, polypeptides, proteins, or nucleic acids found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of peptides, polypeptides, proteins, or nucleic acids are intended herein. Accordingly, all sequences of peptides, polypeptides, proteins, or nucleic acids found in or derived from nature are considered “native”. The terms encompass the peptides, polypeptides, proteins, or nucleic acids when forming a part of a living organism, organ, tissue or cell, when forming a part of a biological sample, as well as when at least partly isolated from such sources. The terms also encompass peptides, polypeptides, proteins, or nucleic acids when produced by recombinant or synthetic means.

In certain embodiments, peptides, polypeptides, proteins, or nucleic acids, may be human, i.e., their primary sequence may be the same as a corresponding primary sequence of or present in a naturally occurring human peptides, polypeptides, proteins, or nucleic acids. Hence, the qualifier “human” in this connection relates to the primary sequence of the respective peptides, polypeptides, proteins, or nucleic acids, rather than to their origin or source. For example, such peptides, polypeptides, proteins, or nucleic acids may be present in or isolated from samples of human subjects or may be obtained by other means (e.g., by recombinant expression, cell-free transcription or translation, or non-biological nucleic acid or peptide synthesis).

Unless otherwise apparent from the context, reference herein to any peptide, polypeptide, protein, or nucleic acid, or fragment thereof may generally also encompass modified forms of said marker, peptide, polypeptide, protein, or nucleic acid, or fragment thereof, such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The terms “DJ-1 inhibitor”, “inhibitor of DJ-1” or “inhibitor” can be used interchangeably herein and refer to any agent that can serve as an inhibitor of DJ-1. The determination of whether or not a substance of interest, e.g. a siRNA, a miRNA, a binding protein, a small molecule or a compound of interest, is an inhibitor of DJ-1 is within the skill of one of ordinary skill in the art.

In particular embodiments, the agent may inhibit DJ-1 either directly or indirectly, preferably directly.

The expression “direct DJ-1 inhibitor” or “agent directly inhibiting DJ-1” as used herein has a meaning as generally accepted within the art and preferably refers to agents binding to the DJ-1 protein or a polynucleotide encoding DJ-1 thereby inhibiting its function or expression, as well as to agents having a direct effect on the function of DJ-1 by binding to a direct target of DJ-1 (e.g. DJ-1 binding molecule) thereby preventing the binding of DJ-1 to said target.

The expression “indirect DJ-1 inhibitor” or “agent indirectly inhibiting DJ-1” as used herein refers to agents that have an inhibiting effect on the expression or the function of DJ-1 as a result of an achieved effect on the expression or function of a further target (e.g. molecule or analyte), which is different from DJ-1 and different from a DJ-1 target (e.g. DJ-1 binding molecule).

An example of how one could determine if a compound is an inhibitor of DJ-1 would be to isolate the DJ-1 protein. For example, the amino acid sequence of the protein that encodes human DJ-1 can have or comprise Uniprot number Q99497-1 (SEQ ID NO: 3). The protein can be isolated from cells where the DJ-1 is naturally expressed or where it has been overexpressed by means of transfection of a genetic construct or infection with a virus that directs the expression of the DJ-1. The nucleic acid sequence of mRNA that encodes DJ-1 can have or comprise NCBI Reference Sequence NM_007262.5 (“transcript variant 1”, SEQ ID NO: 1) or NM_001123377.1 (“transcript variant 2”, SEQ ID NO: 2). Also mRNA can be isolated from a cell and e.g. be expressed in a host cell. DJ-1 can for example be expressed by recombinant techniques.

An inhibitor to DJ-1 may be effective in any possible way. For example, the expression of DJ-1 (e.g. of DJ-1 protein, mRNA or even transcription of DNA) may be inhibited/reduced. Another possibility can be that the function of DJ-1 may be inhibited/reduced/decreased. In general, any reduction in expression as described herein can be measured by any technique, which is known to the skilled person. For example, such measurement can be performed by “real-time PCR” or “Real-time Polymerase Chain Reaction (RT-PCR)” or qPCR. This technique has the ability to monitor the progress of the PCR as it occurs (i.e., in real time). Data is therefore collected throughout the PCR process, rather than at the end of the PCR. In real-time PCR, reactions are characterized by the point in time during cycling when amplification of a target is first detected rather than the amount of target accumulated after a fixed number of cycles. There are two main methods used to perform quantitative PCR: dye-based and probe-based detection. Both methods rely on calculating the initial (zero cycle) DNA concentration by extrapolating back from a reliable fluorescent signal. The basic principle of this method is known in the art (Arya et al., Expert Rev. Mol. Diagn. 5(2):209-219).

An inhibitor of DJ-1 can thus decrease the expression of a nucleic acid molecule comprising or consisting of SEQ ID NO: 1 or SEQ ID NO: 2 and/or of amino acid sequence comprising or consisting of SEQ ID NO: 3 or a nucleic acid molecule or amino acid sequence having at least 60%, 70%, 80%, 90%, 95%, 96%, 97%, 98% or 99% sequence identity to any of SEQ ID NO: 1, SEQ ID NO: 2 and/or SEQ ID NO: 3, e.g. in a cell compared to a control or compared to the expression before the addition of the DJ-1 inhibitor.

An inhibitor of DJ-1 may additionally or alternatively inhibit/reduce/decrease DJ-1 (function) by 10%, 20%, 30%, 40%, 50%, 60%, 70%, 80%, 90% or more when compared to the activity of DJ-1 without the addition of the inhibitor or compared to the activity of DJ-1 before the addition of the inhibitor. A complete inhibition or block of DJ-1 (function) is present when the enzymatic activity of DJ-1 is inhibited by 100% when compared to the enzymatic activity of DJ-1 without the addition of the inhibitor or compared to the activity of DJ-1 before the addition of the inhibitor.

Upon isolating the DJ-1 protein a person of ordinary skill in the art can measure its activity in the presence or absence of a potential DJ-1 inhibitor, preferably using positive and/or negative controls. Notably, DJ-1 under an oxidative condition can inhibit the aggregation of α-synuclein via its chaperone activity, thus can function as a redox-sensitive chaperone and as a sensor for oxidative stress. Accordingly, DJ-1 can protect neurons against oxidative stress and cell death. Additionally or alternatively, DJ-1 protein can act as a positive regulator of androgen receptor-dependent transcription. Notably, Tregs can be immunosuppressive and can suppress or downregulate induction and proliferation of effector T cells.

Therefore, if the activity of DJ-1 is less in the presence of an alleged inhibitor than in the absence of the alleged inhibitor, then this inhibitor truly is a DJ-1 inhibitor. Then the inhibitor decreases DJ-1 function.

To further confirm that an molecule of interest, compound, small molecule or binding protein as described herein is a DJ-1 inhibitor useful to treat or prevent one or more of the diseases or conditions as taught herein, the inhibitor may be tested in a routine immune cell proliferation assay.

In certain embodiments, the DJ-1 inhibitor is directed to DJ-1 or a polynucleotide encoding DJ-1. In certain embodiments, the DJ-1 inhibitor interacts with DJ-1 or a polynucleotide encoding DJ-1. In certain embodiments, the DJ-1 inhibitor binds to DJ-1 or a polynucleotide encoding DJ-1.

The term “bind” or “interact” as used throughout this specification means that an agent (e.g. the inhibitor as described herein) binds to or influences one or more desired molecules or analytes (e.g. DJ-1 or a polynucleotide encoding DJ-1). The term “bind” or “interact” does not require that an agent binds the one or more desired molecules or analytes substantially to the exclusion of other molecules.

In certain embodiments, the DJ-1 inhibitor is specifically directed to DJ-1 or a polynucleotide encoding DJ-1. In certain embodiments, the DJ-1 inhibitor specifically interacts with DJ-1 or a polynucleotide encoding DJ-1. In certain embodiments, the DJ-1 inhibitor specifically binds to DJ-1 or a polynucleotide encoding DJ-1.

The term “specifically bind” or “specifically interact” as used throughout this specification means that an agent (e.g. the inhibitor as described herein) binds to or influences one or more desired molecules or analytes (e.g. DJ-1 or a polynucleotide encoding DJ-1) substantially to the exclusion of other molecules which are random or unrelated, and optionally substantially to the exclusion of other molecules that are structurally related. The term “specifically bind” or “specifically interact” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

The binding of an agent to a target and the affinity and specificity of said binding may be determined by any methods known in the art. Non-limiting examples thereof include binding competition assays, co-immunoprecipitation, bimolecular fluorescence complementation, affinity electrophoresis, label transfer, phage display, proximity ligation assay (PLA), Tandem affinity purification (TAP), in-silico docking and calculation of the predicted Gibbs binding energy, immunoassays, dual-luciferase reporter assay system, and RNA fluorescence in situ hybridization (FISH).

In certain embodiments of the methods, uses, or products, as taught herein, the inhibitor is one or more agents selected from the group consisting of a chemical substance, an antibody, an antibody fragment, an antibody-like protein scaffold, a protein or polypeptide, a peptide, a peptidomimetic, an aptamer, a photoaptamer, a spiegelmer, a nucleic acid, a gene-editing system, an antisense agent, an RNAi agent, and a soluble receptor. In particular embodiments, the inhibitor is one or more agents selected from the group consisting of an antibody, an antibody fragment, an antibody-like protein scaffold, a nucleic acid, a gene-editing system, an antisense agent, and an RNAi agent.

In particular embodiments, the inhibitor is one or more agents selected from the group consisting of an antibody specifically binding DJ-1, an antibody fragment specifically binding DJ-1, an antibody-like protein scaffold specifically binding DJ-1, a nucleic acid specifically binding a polynucleotide encoding DJ-1, a gene-editing system specifically binding a polynucleotide encoding DJ-1, an antisense agent specifically binding a polynucleotide encoding DJ-1, and an RNAi agent specifically binding a polynucleotide encoding DJ-1.

As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, peptides, polypeptides, proteins, peptidomimetics, antibodies, antibody fragments, antibody-like protein scaffolds, aptamers, photoaptamers, spiegelmers, chemical substances, preferably organic molecules, more preferably small organic molecules, lipids, carbohydrates, polysaccharides, etc., and any combinations thereof. The term “agent” may denote a “therapeutic agent” or “drug”, useful for or used in the treatment, cure, prevention, or diagnosis of a disease or conditions as taught herein.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, the agent may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a heavy-chain antibody, preferably a heavy-chain antibody derived from camelids; (2) by expression of a nucleotide sequence encoding a V_{HH} domain; (3) by “humanization” of a naturally occurring V_{HH} domain or by expression of a nucleic acid encoding a such humanized V_{HH} domain; (4) by “camelization” of a V_{H} domain from any animal species, and in particular from a mammalian species, such as from a human being, or by expression of a nucleic acid encoding such a camelized V_{H} domain; (5) by “camelization” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

In certain embodiments, the antibody may be a polyclonal antibody, a monoclonal antibody, a chimeric antibody, a humanized antibody, a primatized antibody, a human antibody, a Nanobody®, an intrabody, or mixtures thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al., Gebauer and Skerra, Gill and Damle, Skerra 2000, and Skerra 2007, and include without limitation affibodies, based on the Z-domain of staphylococcal protein A, a three-helix bundle of 58 residues providing an interface on two of its alpha-helices (Nygren); engineered Kunitz domains based on a small (ca. 58 residues) and robust, disulphide-crosslinked serine protease inhibitor, typically of human origin (e.g. LACI-D1), which can be engineered for different protease specificities (Nixon and Wood); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra 2008); DARPins, designed ankyrin repeat domains (166 residues), which provide a rigid interface arising from typically three repeated beta-turns (Stumpp et al.); avimers (multimerized LDLR-A module) (Silverman et al.); and cysteine-rich knottin peptides (Kolmar).

The term “aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “soluble receptor” generally refers to the soluble (i.e., circulating, not bound to a cell) form of a cell-surface molecule, e.g., a cell-surface receptor, or a fragment or derivative thereof. For example, a cell-surface molecule can be made soluble by attaching a soluble fusion partner, e.g., an immunoglobulin (Ig) moiety, or a portion thereof, to the extracellular domain, or by removing its transmembrane domain.

Targeted genome modification is a powerful tool for genetic manipulation of cells and organisms, including mammals. Genome modification or gene editing, including insertion, deletion or replacement of DNA in the genome, can be carried out using a variety of known gene editing systems. The term “gene-editing system” or “genome editing system” as used herein refers to a tool to induce one or more nucleic acid modifications, such as DNA or RNA modifications, into a specific DNA or RNA sequence within a cell. Gene editing systems typically make use of an agent capable of inducing a nucleic acid modification. In certain embodiments, the agent capable of inducing a nucleic acid modification may be a (endo)nuclease or a variant thereof having altered or modified activity. (endo)Nucleases typically comprise programmable, sequence-specific DNA- or RNA-binding modules linked to a nonspecific DNA or RNA cleavage domain. In DNA, these nucleases create site-specific double-strand breaks at desired locations in the genome. The induced double-stranded breaks are repaired through non-homologous end-joining or homologous recombination, resulting in targeted mutations. In certain embodiments, said (endo)nuclease may be RNA-guided. In certain embodiments, said (endo)nuclease can be engineered nuclease such as a Clustered Regularly Interspaced Short Palindromic Repeats (CRISPR) associated (Cas) (endo)nuclease, such as Cas9, Cpf1, or C2c2, a (zinc finger nuclease (ZFN), a transcription factor-like effector nuclease (TALEN), a meganuclease, or modifications thereof. Methods for using TALEN technology, Zinc Finger technology and CRISPR/Cas technology are known by the skilled person. Accordingly, in particular embodiments, the inhibitor of DJ-1 is a genome editing system, i.e. a combination of nuclease and RNA guide, wherein said RNA guide targets DJ-1 resulting in a mutation thereof which affects DJ-1 function.

In certain embodiments of the methods, uses, or products, as taught herein, the inhibitor of DJ-1 may be a chemical substance. In certain embodiments of the methods, uses, or products, as taught herein, the inhibitor of DJ-1 may be a chemical substance, wherein the chemical substance is an organic molecule. Preferably, the inhibitor of DJ-1 is a small organic molecule. In certain embodiments, the inhibitor of DJ-1 is a chemical substance, wherein the chemical substance is a small molecule.

The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

In certain embodiments of the methods, uses, or products, as taught herein, the inhibitor of DJ-1 is a nucleic acid. In certain embodiments of the methods, uses, or products, as taught herein, the inhibitor is a nucleic acid, wherein the nucleic acid is an oligonucleotide.

The term “oligonucleotide” as used herein refers to a nucleic acid (including nucleic acid analogues and mimetics) oligomer or polymer as defined herein. Preferably, an oligonucleotide is (substantially) single-stranded. Oligonucleotides as intended herein may be preferably between about 10 and about 100 nucleoside units (i.e., nucleotides or nucleotide analogues) in length, preferably between about 15 and about 50, more preferably between about 20 and about 40, also preferably between about 20 and about 30. Preferably, oligonucleotides as intended herein may comprise one or more or all non-naturally occurring heterocyclic bases and/or one or more or all non-naturally occurring sugar groups and/or one or more or all non-naturally occurring inter-nucleoside linkages, the inclusion of which may improve properties such as, for example, enhanced cellular uptake, increased stability in the presence of nucleases and increased hybridization affinity, increased tolerance for mismatches, etc. Further, oligonucleotides as intended herein may be configured to not activate RNAse H, accordance with known techniques (e.g. U.S. Pat. No. 5,149,797).

The term “antisense” generally refers to an agent (e.g., an oligonucleotide as defined elsewhere in the specification) configured to specifically anneal with (hybridise to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein may typically be capable of annealing with (hybridising to) the respective target nucleic acid sequences at high stringency conditions, and capable of hybridising specifically to the target under physiological conditions.

The terms “complementary” or “complementarity” as used herein with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The sequence of an antisense agent need not be 100% complementary to that of its target sequence to bind or hybridise specifically with the latter as defined elsewhere in the specification. An antisense agent may be said to be specifically hybridisable when binding of the agent to a target nucleic acid molecule interferes with the normal function of the target nucleic acid such as to attain an intended outcome (e.g., loss of utility), and there is a sufficient degree of complementarity to avoid non-specific binding of the antisense agent to non-target sequences under conditions in which specific binding is desired, i.e., under physiological conditions in the case of in vivo assays or therapeutic treatment, and in the case of in vitro assays, under conditions in which the assays are performed. Thus, “specifically hybridisable” and “complementary” may indicate a sufficient degree of complementarity or precise pairing such that stable and specific binding occurs between an antisense agent and a nucleic acid target.

Preferably, to ensure specificity of antisense agents towards the desired target over unrelated molecules, the sequence of said antisense agents may be at least about 80% identical, preferably at least about 90% identical, more preferably at least about 95% identical, such as, e.g., about 96%, about 97%, about 98%, about 99% and up to 100% identical to the respective target sequence.

Antisense agents as intended herein preferably comprise or denote antisense molecules such as more preferably antisense nucleic acid molecules or antisense nucleic acid analogue molecules. Preferably, antisense agents may refer to antisense oligonucleotides or antisense oligonucleotide analogues.

Antisense agents such as oligonucleotides as taught herein may be further conjugated (e.g., covalently or non-covalently, directly or via a suitable linker) to one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given agent to be uniformly modified, and in fact more than one of the aforementioned modifications may be incorporated in a single agent or even at a single nucleoside within an oligonucleotide. Further included are antisense compounds that are chimeric compounds. “Chimeric” antisense compounds or “chimeras” are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

The antisense agent or RNA guide can, for example, be designed with regard to a target sequence. The target sequence can, for example, be a nucleic acid molecule of any of SEQ ID NO: 1 and SEQ ID NO: 2. The nucleic acid molecule that can be used in the present invention can therefore comprise a sequence that is complementary to a sequence that comprises any of SEQ ID NO: 1 and SEQ ID NO: 2. The present invention also encompasses nucleic acid sequences (in particular siRNA sequences) which are 50%, 60%, 70%, 80%, 85%, 90%, 95%, 96%, 97%, 98%, 99% or 100% complementary to a nucleic acid molecule that comprises a sequence of any of SEQ ID NO: 1 and SEQ ID NO: 2.

The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules.

The term “RNA interference agent” or “RNAi agent” refers to ribonucleic acid sequences, modified ribonucleic acid sequences, or DNA sequences encoding said ribonucleic acid sequences, which cause RNA interference and thus decrease expression of the target gene.

An RNAi (RNA interference) agent typically comprises, consists essentially of or consists of a double-stranded portion or region (notwithstanding the optional and potentially preferred presence of single-stranded overhangs) of annealed complementary strands, one of which has a sequence corresponding to a target nucleotide sequence (hence, to at least a portion of an mRNA) of the target gene to be down-regulated. The other strand of the RNAi agent is complementary to said target nucleotide sequence. Non-limiting examples of RNAi agents are shRNAs, siRNAs, miRNAs, and DNA-RNA hybrids.

Whereas the sequence of an RNAi agent need not be completely identical to a target sequence to be down-regulated, the number of mismatches between a target sequence and a nucleotide sequence of the RNAi agent is preferably no more than 1 in 5 bases, or 1 in 10 bases, or 1 in 20 bases, or 1 in 50 bases.

Preferably, to ensure specificity of RNAi agents towards the desired target over unrelated molecules, the sequence of said RNAi agents may be at least about 80% identical, preferably at least about 90% identical, more preferably at least about 95% identical, such as, e.g., about 96%, about 97%, about 98%, about 99% and up to 100% identical to the respective target sequence.

An RNAi agent may be formed by separate sense and antisense strands or, alternatively, by a common strand providing for fold-back stem-loop or hairpin design where the two annealed strands of an RNAi agent are covalently linked.

An siRNA molecule may be typically produced, e.g., synthesised, as a double stranded molecule of separate, substantially complementary strands, wherein each strand is about 18 to about 35 bases long, preferably about 19 to about 30 bases, more preferably about 20 to about 25 bases and even more preferably about 21 to about 23 bases.

shRNA is in the form of a hairpin structure. shRNA can be synthesized exogenously or can be formed by transcribing from RNA polymerase III promoters in vivo. Preferably, shRNAs can be engineered in host cells or organisms to ensure continuous and stable suppression of a desired gene. It is known that siRNA can be produced by processing a hairpin RNA in cells.

RNAi agents as intended herein may include any modifications as set out herein for nucleic acids and oligonucleotides, in order to improve their therapeutic properties.

In embodiments, at least one strand of an RNAi molecules may have a 3′ overhang from about 1 to about 6 bases in length, e.g., from 2 to 4 bases, more preferably from 1 to 3 bases. For example, one strand may have a 3′ overhang and the other strand may be either blunt-ended or may also have a 3′ overhang. The length of the overhangs may be the same or different for each strand. The 3′ overhangs can be stabilised against degradation. For example, the RNA may be stabilised by including purine nucleotides, such as A or G nucleotides. Alternatively, substitution of pyrimidine nucleotides by modified analogues, e.g., substitution of U 3′ overhangs by 2′-deoxythymidine is tolerated and does not affect the efficiency of RNAi.

An exemplary but non-limiting siRNA molecule may by characterized by any one or more, and preferably by all of the following criteria:
at least about 80% sequence identity, more preferably at least about 90% or at least about 95% or
at least about 97% sequence identity to target mRNA;
having a sequence which targets an area of the target gene present in mature mRNA (e.g., an exon or alternatively spliced intron);
showing a preference for targeting the 3′ end of the target gene.

The exemplary siRNA may be further characterised by one or more or all of the following criteria:
having a double-stranded nucleic acid length of between 16 to 30 bases and preferably of between 18 to 23 bases, and preferably of 19 nucleotides;
having GC content between about 30 and about 50%
having a TT(T) sequence at 3′ end;
showing no secondary structure when adopting the duplex form;
having a Tm (melting temperature) of lower than 20° C.
having the nucleotides indicated here below in the sequence of the nucleotides, wherein “h” is A, C, T/U but not G; wherein “d” is A, G, T/U but not C, and wherein “w” is A or T/U, but not G or C:

| | | — | — | 1 | 2 | 3 | 4 | 5 | 6 | 7 | 8 | 9 | 10 | 11 | 12 | 13 | 14 | 15 | 16 | 17 | 18 | 19 | — | — | |
|---|---|---|---|---|---|---|---|---|---|---|---|---|---|---|---|---|---|---|---|---|---|---|---|---|---|
| mRNA | P′5 | A | A | | | A | | | | | | | U | | | h | | | | | | w | | | 3′-OH |
| si-ASense | OH-3′ | T | T | | | U | | | | | | | A | | | d | | | | | | w | | | 5′-P |
| si-Sense | P-5′ | | | | | A | | | | | | | U | | | h | | | | | | w | T | T | 3′-OH |

RNAi agents as intended herein may particularly comprise or denote (i.e., may be selected from a group comprising or consisting of) RNAi nucleic acid molecules or RNAi nucleic acid analogue molecules, such as preferably short interfering nucleic acids and short interfering nucleic acid analogues (siNA) such as short interfering RNA and short interfering RNA analogues (siRNA), and may further denote inter alia double-stranded RNA and double-stranded RNA analogues (dsRNA), micro-RNA and micro-RNA analogues (miRNA), and short hairpin RNA and short hairpin RNA analogues (shRNA).

Production of antisense agents and RNAi agents can be carried out by any processes known in the art, such as inter alia partly or entirely by chemical synthesis (e.g., routinely known solid phase synthesis; an exemplary an non-limiting method for synthesising oligonucleotides on a modified solid support is described in U.S. Pat. No. 4,458,066; in another example, diethyl-phosphoramidites are used as starting materials and may be synthesised as described by Beaucage et al. 1981 (Tetrahedron Letters 22: 1859-1862)), or partly or entirely by biochemical (enzymatic) synthesis, e.g., by in vitro transcription from a nucleic acid construct (template) using a suitable polymerase such as a T7 or SP6 RNA polymerase, or by recombinant nucleic acid techniques, e.g., expression from a vector in a host cell or host organism. Nucleotide analogues can be introduced by in vitro chemical or biochemical synthesis. In an embodiment, the antisense agents of the invention are synthesised in vitro and do not include antisense compositions of biological origin, or genetic vector constructs designed to direct the in vivo synthesis of antisense molecules.

The inhibitor of DJ-1 for use as taught herein can be a nucleic acid molecule, such as a siRNA, that has a sequence identity of at least 50%, 60%, 70%, 80%, 90%, 95%, 96%, 97%, 98%, 99% or 100% to SEQ ID NO: 4.

The anti-DJ-1 siRNA sequence of SEQ ID NO: 4 is as follows: AATGGAGGTCATTACACCTAC.

In certain embodiments of the methods, uses, or products, as taught herein, the inhibitor is an RNAi agent, wherein the RNAi agent, preferably a siRNA, has a sequence identity of at least 50%, 60%, 70%, 80%, 90%, 95%, 96%, 97%, 98%, 99% or 100% to SEQ ID NO: 4.

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62 (Henikoff et al., 1992, Proc. Natl. Acad. Sci., 89:10915-10919), cost to open a gap=11, cost to extend a gap=1, expectation value=10.0, word size=3).

An example procedure to determine the percent identity between a particular amino acid sequence and the amino acid sequence of a query polypeptide will entail aligning the two amino acid sequences using the Blast 2 sequences (Bl2seq) algorithm, available as a web application or as a standalone executable programme (BLAST version 2.2.31+) at the NCBI web site (www.ncbi.nlm.nih.gov), using suitable algorithm parameters. An example of suitable algorithm parameters include: matrix=Blosum62, cost to open a gap=11, cost to extend a gap=1, expectation value=10.0, word size=3). If the two compared sequences share homology, then the output will present those regions of homology as aligned sequences. If the two compared sequences do not share homology, then the output will not present aligned sequences. Once aligned, the number of matches will be determined by counting the number of positions where an identical amino acid residue is presented in both sequences. The percent identity is determined by dividing the number of matches by the length of the query polypeptide, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by Bl2seq already conveniently includes the percentage of identities.

In certain embodiments of the methods, uses, or products, as taught herein, the inhibitor of DJ-1, such as a nucleic acid or the RNAi agent, may be provided within a plasmid vector.

The terms “plasmid vector”, “expression vector” or “vector” as used herein refers to nucleic acid molecules, typically DNA, to which nucleic acid fragments, preferably the recombinant nucleic acid molecule as defined herein, may be inserted and cloned, i.e., propagated. Hence, a vector will typically contain one or more unique restriction sites, and may be capable of autonomous replication in a defined cell or vehicle organism such that the cloned sequence is reproducible. A vector may also preferably contain a selection marker, such as, e.g., an antibiotic resistance gene, to allow selection of recipient cells that contain the vector. Vectors may include, without limitation, plasmids, phagemids, bacteriophages, bacteriophage-derived vectors, PAC, BAC, linear nucleic acids, e.g., linear DNA, transposons, viral vectors, etc., as appropriate (see, e.g., Sambrook et al., 1989; Ausubel 1992). Viral vectors may include inter alia retroviral vectors, lentiviral vectors, adenoviral vectors, or adeno-associated viral vectors, for example, vectors based on HIV, SV40, EBV, HSV or BPV. Expression vectors are generally configured to allow for and/or effect the expression of nucleic acids or open reading frames introduced thereto in a desired expression system, e.g., in vitro, in a cell, organ and/or organism. For example, expression vectors may advantageously comprise suitable regulatory sequences.

Factors of importance in selecting a particular vector include inter alia: choice of recipient cell, ease with which recipient cells that contain the vector may be recognised and selected from those recipient cells which do not contain the vector; the number of copies of the vector which are desired in particular recipient cells; whether it is desired for the vector to integrate into the chromosome or to remain extra-chromosomal in the recipient cells; and whether it is desirable to be able to “shuttle” the vector between recipient cells of different species.

Expression vectors can be autonomous or integrative. A nucleic acid can be in introduced into a cell in the form of an expression vector such as a plasmid, phage, transposon, cosmid or virus particle. The recombinant nucleic acid can be maintained extrachromosomally or it can be integrated into the cell chromosomal DNA. Expression vectors can contain selection marker genes encoding proteins required for cell viability under selected conditions (e.g., URA3, which encodes an enzyme necessary for uracil biosynthesis, or LEU2, which encodes an enzyme required for leucine biosynthesis, or TRP1, which encodes an enzyme required for tryptophan biosynthesis) to permit detection and/or selection of those cells transformed with the desired nucleic acids. Expression vectors can also include an autonomous replication sequence (ARS). The ARS may comprise a centromere (CEN) and an origin of replication (ORI). For example, the ARS may be ARS18 or ARS68.

Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the cell species to be transformed. A nucleotide sequence containing a nucleic acid of interest for expression is inserted in this vector between the first and second insertable DNA fragments, whether before or after the marker gene. Integrative vectors can be linearized prior to transformation to facilitate the integration of the nucleotide sequence of interest into the cell genome.

As used herein, the term “promoter” refers to a DNA sequence that enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

An “operable linkage” is a linkage in which regulatory sequences and sequences sought to be expressed are connected in such a way as to permit said expression. For example, sequences, such as, e.g., a promoter and an open reading frame (ORF), may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence. Hence, “operably linked” may mean incorporated into a genetic construct so that expression control sequences, such as a promoter, effectively control transcription/expression of a sequence of interest.

The promotor may be a constitutive or inducible (conditional) promoter. A constitutive promoter is understood to be a promoter whose expression is constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues. Non-limiting examples of promoters include T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter.

Prior to introducing the vectors into a cell of interest, the vectors can be grown (e.g., amplified) in bacterial cells such as Escherichia coli (E. coli). The vector DNA can be isolated from bacterial cells by any of the methods known in the art which result in the purification of vector DNA from the bacterial milieu. The purified vector DNA can be extracted extensively with phenol, chloroform, and ether, to ensure that no E. coli proteins are present in the plasmid DNA preparation, since these proteins can be toxic to mammalian cells.

In certain embodiments of the methods, uses, or products, as taught herein, the inhibitor of DJ-1, such as the nucleic acid or the RNAi agent, may be modified or encapsulated by synthetic or natural nanoparticles; preferably wherein the nanoparticle is a liposomal nanoparticle.

In certain embodiments, the inhibitor for use, such as the nucleic acid molecule like siRNA or miRNA, are provided within a plasmid vector and/or are modified or encapsulated by synthetic or natural nanoparticles. The synthetic or natural lipid nanoparticles may for example comprise lipids as well as polymers and/or metals. The nanoparticle may comprise one or more of natural or synthetic lipids (e.g., liposomes, micelles), polymers (e.g. chitosan, poly(lactic-co-glycolic) acid (PLGA), polylactic acid (PLA), polyethilenimine (PEI), atelocollagen), carbon nanotubes, quantum dots, gold nanoshells or iron oxide magnetic. The nanoparticle can additionally or alternatively be biodegradable. For example, the nanoparticle can be a liposomal nanoparticle. Exemplary liposomal nanoparticles include, but are not limited to cationic-lipid based liposome, neutral lipid-based nanoliposome, a solid lipid-based systems (SNALP and SLN) or lipidoid nanoparticle.

In certain embodiments, the DJ-1 inhibitor as taught herein may increase the amount, e.g. the number or percentage, of total CD8 T cells, such as in peripheral lymph nodes and/or the spleen of an elderly subject. In certain embodiments, the DJ-1 inhibitor as taught herein may increase the amount, e.g. the number or percentage, of naïve non-senescent CD8 T cells, such as CD45RO⁻CD28⁺, CD45RO⁻CD27⁺, CD45RO⁻CD57⁻ and CD27⁺CD28⁺ CD8 T cells.

In certain embodiments, the DJ-1 inhibitor as taught herein may increase the amount, e.g. the number or percentage, of naïve CD4 T cells. In certain embodiments, the DJ-1 inhibitor as taught herein may increase the amount, e.g. the number or percentage, of CD4 and/or CD8 naïve CD44^{low}CD62L^{high} T cells, such as in spleen, peripheral blood and/or peripheral lymph nodes of an elderly subject.

In certain embodiments, the DJ-1 inhibitor as taught herein may decrease the amount, e.g. the number or percentage, of CD4 and/or CD8 CD44^{high}CD62L^{low} T effector memory cells, such as in spleen, peripheral blood and/or peripheral lymph nodes of an elderly subject.

In certain embodiments, the DJ-1 inhibitor as taught herein may decrease the amount, e.g. the number or percentage, of CD8 central memory T cells, such as in spleen, peripheral blood and/or peripheral lymph nodes of an elderly subject.

In certain embodiments, the DJ-1 inhibitor as taught herein may decrease the amount, e.g. the number or percentage, of positive cells for one or more of the critical proliferation, exhaustion and activation markers, such as Ki67, PD-1, CTLA4, CD69, ICOS and Helios, in total CD4 and/or CD8 T cells, such as in spleens and/or lymph nodes of an elderly subject.

In the aforementioned embodiments, the change, e.g. increase or reduction (decrease), may be defined relative to (i.e. compared to) age- and/or gender-matched subjects which have not been treated with the DJ-1 inhibitor.

In certain embodiments, the DJ-1 inhibitor as taught herein may increase the amount, e.g. the number or percentage, of non-exhausted CD8 T cells, e.g. PD-1⁻Tbet⁻ and PD-1⁻Eomes⁻ CD8 T cells. In certain embodiments, the DJ-1 inhibitor as taught herein may decrease the amount, e.g. the number or percentage, of PD-1-expressing CD4 T cells, e.g. of CD45RO⁺ T cells. In certain embodiments, the DJ-1 inhibitor as taught herein may decrease the amount, e.g. the number or percentage, of FOXP3⁺CD4⁺ Tregs. In the aforementioned embodiments, the change, e.g. increase or reduction (decrease), in the amount, e.g. the number or percentage, of T cells may be defined relative to (age- and/or gender-matched if not mentioned otherwise) subjects which have not been treated with the DJ-1 inhibitor.

A molecule or marker (e.g. cell surface marker) such as a peptide, polypeptide, protein, or nucleic acid, or a group of two or more molecules or markers such as two or more peptides, polypeptides, proteins, or nucleic acids, is “measured” in a sample when the presence or absence and/or quantity of said molecule or marker or of said group of molecules or markers is detected or determined in the sample, preferably substantially to the exclusion of other molecules and markers. The terms “quantity”, “amount” and “level” are synonymous and generally well-understood in the art. The terms as used herein may particularly refer to an absolute quantification of a number of cells, a peptide, polypeptide, protein, or nucleic acid in a sample, or to a relative quantification of a number of cells, a peptide, polypeptide, protein, or nucleic acid in a sample, i.e., relative to another value such as relative to a reference value as taught herein.

The terms “percentage” or “frequency” as used herein to a relative quantification of a number of cells in a sample, i.e. a quantification of a certain subset of cells (e.g. total CD8⁺ T cells) in a sample relative to the quantification of all cells in the sample.

The quantity of a peptide, polypeptide or protein may also be represented by the activity of a peptide, polypeptide or protein. Activity of peptide, polypeptide or protein in a sample may also be expressed in absolute terms, e.g., in enzymatic units per volume, or relative terms.

In certain embodiments, the DJ-1 inhibitor as taught herein may decrease the mRNA expression of one or more exhaustion genes, such as LAG3 and TIM3/HAVCR2, in CD8 T cells. In certain embodiments, the DJ-1 inhibitor as taught herein may decrease the mRNA expression of one or more senescence related genes, such as CD57/B3GAT1, CD85j/LILRB1 and KLRG1, in CD8 T cells. In certain embodiments, the DJ-1 inhibitor as taught herein may increase the mRNA expression of costimulatory genes CD28 and/or CD27, in CD8 T cells. In certain embodiments, the DJ-1 inhibitor as taught herein may decrease the mRNA expression of one or more NK cell-related markers, such as KIR3DX1, KLRD1/CD94 and KLRF1/NKP80, in CD8 T cells. In certain embodiments, the DJ-1 inhibitor as taught herein may decrease the mRNA expression of one or more Cip/Kip family members including CDKN1A/p21, CDKN1B/p27, and CDKN1C/p57. In certain embodiments, the DJ-1 inhibitor as taught herein may decrease the mRNA expression of one or more INK4 family members including CDKN2C/p18 and CDKN2D/p19. In the foregoing embodiments, the change, e.g. increase or reduction (decrease), of the mRNA expression may be defined relative to (age- and/or gender-matched) subjects which have not been treated with the DJ-1 inhibitor.

The terms “subject”, “individual” or “patient” are used interchangeably throughout this specification, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. In certain preferred embodiments, the subject is a human subject. In other embodiments, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

In certain embodiments, the subject may have been selected (e.g. diagnosed) to have or may have a premature aging disease, such as Hutchinson-Gilford progeria syndrome (HGPS), Bloom syndrome (BS), Cockayne syndrome (CS), Mandibuloacral Dysplasia with Type A Lipodystrophy (MADA); Werner syndrome (WS), Rothmund-Thomson syndrome (RTS), Seip syndrome, xeroderma pigmentosum (XP), trichothiodystrophy (TTD), combined xeroderma pigmentosum-Cockayne syndrome (XP-CS), restrictive dermopathy (RD), Nijmegen Breakage Syndrome, and ataxia-telangiectasia. Preferably, the subject may have been selected (e.g. diagnosed) to have or may have Hutchinson-Gilford progeria syndrome (HGPS), Bloom syndrome (BS), Cockayne syndrome (CS), Mandibuloacral Dysplasia with Type A Lipodystrophy (MADA); Werner syndrome (WS), Rothmund-Thomson syndrome (RTS), Seip syndrome, xeroderma pigmentosum (XP), trichothiodystrophy (TTD), combined xeroderma pigmentosum-Cockayne syndrome (XP-CS), or restrictive dermopathy (RD). More preferably, the subject may have been selected (e.g. diagnosed) to have or may have HGPS.

In certain embodiments of the methods or uses, as taught herein, the subject, e.g. a subject having been selected (e.g. diagnosed) to have or having a premature aging disease, may be a fetus (e.g. from nine weeks after fertilization until birth), an infant (e.g. having an age of 0 to 12 months), toddler (e.g. having an age of 12 months to 36 months), child (e.g. having an age of 3 to 10 years), preadolescent (e.g. having an age of 10 to 13 years), adolescent (e.g. having an age of 13 to 18 years), or adult (e.g. having an age of at least 18 years, such as having an age of 18 years to 59 years).

In certain embodiments of the methods or uses, as taught herein, the subject, e.g. a subject having been selected (e.g. diagnosed) to have or having a premature aging disease, may have an age of 0 (i.e. birth) to 59 years. For instance, the subject, e.g. a subject having been selected (e.g. diagnosed) to have or having a premature aging disease, may have an age of 0 (i.e. birth) to 18 years, an age of 0 (i.e. birth) to 13 years, or an age of 0 (i.e. birth) to 10 years.

In certain embodiments of the methods or uses, as taught herein, the subject may be an elderly subject.

The term “elderly subject” refers to a subject of old age, i.e. the ages nearing or surpassing the life expectancy of a subject, preferably a human subject. For example, an elderly subject as defined herein may be an elderly subject according to the definition of the World Health Organization (WHO) or according to the definition of the United Nations (UN).

In certain embodiments of the methods or uses, as taught herein, the subject has an age of at least 60 years. Preferably, the subject has an age of at least 65 years. In certain embodiments, the subject has an age of at least 70 years, at least 75 years, at least 80 years, at least 85 years, at least 90 years, at least 95 years or more.

In certain embodiments of the methods or uses, as taught herein, the subject belongs to the subgroup of the young old (60 to 69 years), the middle old (70 to 79 years), or the very old (80 or more years).

In certain embodiments, the subject may be a non-diseased or a diseased subject. A subject, in particular an elderly subject, may not (yet) have any symptoms of an immunoaging-related disease but may benefit from the treatment with an inhibitor of DJ-1 as taught herein in order to lessen or stop the age-related decline of the immune system and/or to rejuvenate the immune system.

In certain embodiments, said subject is a patient at risk of immunoaging, such as a patient with accumulated exposure to infectious agents, autoantigens and/or cancer antigens, or a patient having suffered from malnutrition.

In certain embodiments, the subject may be a subject at risk of developing a premature aging disease, an immunoaging-related disease, and/or vaccination inefficiency.

In certain embodiments, the subject may be a subject being treated for or in need of treatment of immunoaging, a premature aging disease, an immunoaging-related disease, and/or vaccination inefficiency.

In certain embodiments, the subject may have been selected (e.g. diagnosed) to have or may have one or more of:
a decreased amount, e.g. a decreased number/frequency, of naïve non-senescent CD8 T cells, such as CD45RO⁻CD28⁺, CD45RO⁻CD27⁺, CD45RO⁻CD57⁻ and/or CD27⁺CD28⁺CD8 T cells;
an increased amount, e.g. an increased number/frequency, of exhausted CD8 T cells;
an increased amount, e.g. an increased number/frequency, of PD-1-expressing CD4 T cells, e.g. of CD45RO⁺ T cells;
a decreased frequency/number of CD45RA⁺CD45RO⁺ naïve CD4 and/or CD8 T cells; and/or
an increased amount, e.g. an increased number/frequency, of FOXP3⁺CD4⁺ Tregs;

relative to (i.e. compared to) the number of the respective T cells in age- and/or gender-matched (control) subjects or relative to (i.e. compared to) the number of the respective T cells measured in the subject at an earlier time point (e.g. time point earlier in the lifespan of the subject such as in the adult subject). The criteria listed above are markers for immunoaging.

In certain embodiments, the subject may have been selected (e.g. diagnosed) to have or may have one or more of, such as all of:
a decreased amount, e.g. a decreased number/frequency, of naïve non-senescent CD8 T cells, such as CD45RO⁻CD28⁺, CD45RO⁻CD27⁺, CD45RO⁻CD57⁻ and/or CD27⁺CD28⁺CD8 T cells;
an increased amount, e.g. an increased number/frequency, of exhausted CD8 T cells;
an increased amount, e.g. an increased number/frequency, of PD-1-expressing CD4 T cells, e.g. of CD45RO⁺ T cells; and
an increased amount, e.g. an increased number/frequency, of FOXP3⁺CD4⁺ Tregs;

relative to (i.e. compared to) the number of the respective T cells in age- and/or gender-matched (control) subjects or relative to (i.e. compared to) the number of the respective T cells measured in the subject at an earlier time point (e.g. time point earlier in the lifespan of the subject such as in the adult subject). The criteria listed above are markers for immunoaging.

In certain embodiments, the subject may have been selected (e.g. diagnosed) to have or may have one or more of:
an increased mRNA expression of one or more exhaustion genes, such as LAG3 and/or TIM3, in CD8 T cells;
an increased mRNA expression of one or more senescence related genes, such as CD57, CD85j and/or KLRG1, in CD8 T cells;
a decreased mRNA expression of costimulatory genes, such as CD28 and/or CD27, in CD8 T cells;
an increased mRNA expression of one or more NK cell-related markers, such as KIR3DX1, KLRD1/CD94 and/or KLRF1/NKP80, in CD8 T cells;
an increased mRNA expression of one or more Cip/Kip family members, such as CDKN1A/p21, CDKN1B/p27, and/or CDKN1C/p57, in CD8 T cells; and
an increased mRNA expression of one or more INK4 family members, such as CDKN2C/p18 and/or CDKN2D/p19, in CD8 T cells;

relative to (i.e. compared to) the mRNA expression of the respective markers in CD8 T cells of age- and/or gender-matched (control) subjects or relative to (i.e. compared to) the mRNA expression of the respective markers in CD8 T cells measured (or observed) in said subject at an earlier time point. In certain embodiments, the CD8 T cells may be isolated from peripheral blood. It will be understood to the skilled person that, one or more of the criteria described above can be used as an indication to determine whether or not the subject is suffering from immunoaging. Accordingly, the criteria listed above are markers for immunoaging.

Thus, in particular embodiments, the subject may be an elderly subject having one or more of the T cell profiles or mRNA expression profiles described above. However, in particular embodiments, the patient is not elderly but is characterized by one or more of the T cell profiles or mRNA expression profiles described above, e.g. a subject having been selected (e.g. diagnosed) to have or having a premature aging disease. In particular embodiments, the patient has not been diagnosed with an autoimmune disease, allergy, an infectious disease, or cancer.

As used throughout this specification, the terms “therapy” or “treatment” refer to the alleviation or measurable lessening of one or more symptoms or measurable markers of a pathological condition such as a disease or disorder. The terms encompass primary treatments as well as neo-adjuvant treatments, adjuvant treatments and adjunctive therapies. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments.

As mentioned above, an aspect relates to an inhibitor of DJ-1 for use in a method of treatment (including prevention) of immunoaging in a subject, preferably in an elderly subject. In particular embodiments, said subject is a patient at risk of immunoaging, such as a patient with accumulated exposure to infectious agents, autoantigens and/or cancer antigens, or a patient having suffered from malnutrition.

Accordingly, other aspects provides:
a method of treating (or preventing) immunoaging in a subject, preferably in an elderly subject, comprising administering to the subject a therapeutically or prophylactically effective amount of an inhibitor of DJ-1.
the use of an inhibitor of DJ-1 for the manufacture of a medicament for use in treatment (or prevention) of immunoaging.
the use of an inhibitor of DJ-1 for the treatment (or prevention) of immunoaging.

The present invention advantageously allows to diminish or prevent the age-associated deterioration of the immune system in subjects, particularly elderly subjects. Such subjects treated as taught herein may be less prone to develop an immunoaging-related disease, or may have reduced symptoms and/or slower progression of the immunoaging-related disease.

The terms “immunosenescence” or “immunoaging” refer to a multifactorial condition due to the gradual deterioration of the immune system, such as brought on by natural age advancement or by premature aging diseases. Immunosenescence may lead to many pathologically significant health problems in the aged population.

In certain embodiments, treating (or preventing) immunoaging may comprise one or more of decreasing the genesis of natural Treg cells, decreasing the number of memory T cells, increasing the number of naïve T cells, decreasing T cell exhaustion and senescence, and increasing antigen-specific responses.

In certain embodiments, the method of treating (or preventing) immunoaging comprises a step of diagnosing a subject with immunoaging comprising determining the presence of one or more of the criteria (e.g. markers) of immunoaging as described elsewhere herein.

Provided herein in other embodiments or aspects are:
an inhibitor of DJ-1 for use in rejuvenating the immune system in a subject.
a method of rejuvenating the immune system in a subject, comprising administering to the subject a therapeutically or prophylactically effective amount of an inhibitor of DJ-1.
the use of an inhibitor of DJ-1 for the manufacture of a medicament for use in rejuvenating the immune system.
the use of an inhibitor of DJ-1 for rejuvenating the immune system.

The present invention advantageously allows to rejuvenate the immune system in elderly subjects and/or subjects otherwise suffering from immunoaging e.g. a subject having been selected (e.g. diagnosed) to have or having a premature aging disease. Thereby, the present invention allows to prevent or lessen the chances of incidence of an immunoaging-related disease such as cancer or an infectious disease.

The recitations “rejuvenating the immune system”, “renewing the immune system” or “restoring the immune system” or “enhancing the immune system” or “reinvigorating the immune system” or “re-activating the immune system” or “increasing the immune responsiveness” may be used interchangeably and refer to reverting the state of the immune system to a younger state. In certain embodiments, rejuvenating the immune system may comprise one or more of decreasing the genesis of natural Treg cells, decreasing the number of memory T cells, increasing the number of naïve T cells, decreasing T cell exhaustion and senescence, and increasing antigen-specific responses.

In certain embodiments, the method of rejuvenating the immune system comprises a step of determining whether a subject is in need of rejuvenation of the immune system comprising determining the presence of one or more of the criteria (e.g. markers) of immunoaging as described elsewhere herein.

Signs of an exhausted or senescent immune system have also been observed in patients suffering from a premature aging disease or a disease associated with premature aging.

Accordingly, certain embodiments provide the inhibitor of DJ-1 for use in treating or preventing a premature aging disease in a subject.

A related aspect provides an inhibitor of DJ-1 for use in treating or preventing a premature aging disease in a subject.

The terms “premature aging disease”, “premature aging disorder” or “progeroid syndrome (PS)” refer to a group of rare genetic disorders which mimic physiological aging, making affected individuals appear to be older than they are. Premature aging disorders are typically monogenic, i.e. they arise from mutations of a single gene. Most known PS are due to genetic mutations that lead to either defects in the DNA repair mechanism or defects in lamin A/C. Premature aging diseases also include diseases associated with premature aging, which are diseases which have premature aging as one of their symptoms.

Related embodiments or aspects provide:
a method of treating (or preventing) a premature aging disease in a subject, comprising administering to the subject a therapeutically or prophylactically effective amount of an inhibitor of DJ-1 as taught herein.
the use of an inhibitor of DJ-1 as taught herein for the manufacture of a medicament for use in treatment (or prevention) of a premature aging disease.
the use of an inhibitor of DJ-1 as taught herein for the treatment (or prevention) of a premature aging disease.

In certain embodiments of the methods or uses, as taught herein, the premature aging disease may be selected from the group consisting of Hutchinson-Gilford progeria syndrome (HGPS), Bloom syndrome (BS), Cockayne syndrome (CS), Mandibuloacral Dysplasia with Type A Lipodystrophy (MADA); Werner syndrome (WS), Rothmund-Thomson syndrome (RTS), Seip syndrome, xeroderma pigmentosum (XP), trichothiodystrophy (TTD), combined xeroderma pigmentosum-Cockayne syndrome (XP-CS), restrictive dermopathy (RD), Nijmegen Breakage Syndrome, and ataxia-telangiectasia. Preferably, the premature aging disease may be selected from the group consisting of Hutchinson-Gilford progeria syndrome (HGPS), Bloom syndrome (BS), Cockayne syndrome (CS), Mandibuloacral Dysplasia with Type A Lipodystrophy (MADA); Werner syndrome (WS), Rothmund-Thomson syndrome (RTS), Seip syndrome, xeroderma pigmentosum (XP), trichothiodystrophy (TTD), combined xeroderma pigmentosum-Cockayne syndrome (XP-CS), and restrictive dermopathy (RD). More preferably, the premature aging disease is Hutchinson-Gilford progeria syndrome.

The terms “Hutchinson-Gilford progeria syndrome (HGPS)” or “progeria” can be used interchangeably and refer to an extremely rare autosomal dominant genetic disorder in which symptoms resembling aspects of aging are manifested at a very early age, e.g. at the age of 9 months to 24 months. The cause of progeria may be a point mutation in position 1824 of the lamin A/C (LMNA) gene.

In certain embodiments, the subject may have been selected (e.g. diagnosed) or may have a premature aging disease. In certain embodiments, the subject may be in need of treatment of a premature aging disease.

In certain embodiments, the method of treating a premature aging disease comprises a step of diagnosing a subject with a premature aging disease comprising determining the presence of one or more of the criteria (e.g. markers) of immunoaging as described elsewhere herein.

Certain embodiments provide the inhibitor of DJ-1 for use in treating or preventing an immunoaging-related disease in a subject, wherein the subject has been selected (e.g. diagnosed) to have or has a premature aging disease; or wherein the subject is an elderly subject.

A related aspect provides an inhibitor of DJ-1 for use in treating or preventing an immunoaging-related disease, such as cancer or an infectious disease, in a subject, wherein the subject has been selected (e.g. diagnosed) to have or has a premature aging disease; or wherein the subject is an elderly subject.

Related embodiments or aspects provide:
a method of treating (or preventing) an immunoaging-related disease in a subject, comprising administering to the subject a therapeutically or prophylactically effective amount of an inhibitor of DJ-1 as taught herein, wherein the subject has been selected (e.g. diagnosed) to have or has a premature aging disease; or wherein the subject is an elderly subject.
the use of an inhibitor of DJ-1 as taught herein for the manufacture of a medicament for use in treatment (or prevention) of an immunoaging-related disease.
the use of an inhibitor of DJ-1 as taught herein for the treatment (or prevention) of an immunoaging-related disease.

In certain embodiments, the method of treating an immunoaging-related disease comprises a step of diagnosing a subject with an immunoaging-related disease comprising determining the presence of one or more of the criteria (e.g. markers) of immunoaging as described elsewhere herein. In certain embodiments, the step of diagnosing is performed prior to administering the therapeutically or prophylactically effective amount of an inhibitor of DJ-1.

In certain embodiments, the immunoaging-related disease may be cancer or an infectious disease.

The DJ-1 inhibitors as taught herein can be used to treat or prevent an infectious disease in an elderly subject and/or a subject otherwise suffering from immunoaging, e.g. a subject having been selected (e.g. diagnosed) to have or having a premature aging disease. The phrase “infectious disease” refers to any disease or disorder caused by organisms such as bacteria, viruses, fungi or parasites. The infectious disease can be of viral and/or bacterial origin. Exemplary infectious diseases include but are not limited to multidrug-resistant Acinetobacter baumannii (MDR-Ab), AIDS, Amebiasis, Anaplasmosis, Animal Bites, Animals in Public Settings, Anthrax, Antibiotic Use, Arboviral Encephalitis, Aseptic Meningitis, Avian Influenza, Babesiosis, Baylisascaris (Raccoon Roundworm), Bioterrorism, Bird Flu, Botulism, Brucellosis, Campylobacteriosis, Carbapenem Resistant Enterobacteriaceae (CRE), Catheter Associated Urinary Tract Infection (CAUTI), Central Line-Associated Blood Stream Infection (CLABSI), Chancroid, Chickenpox (Varicella), Chickenpox (Varicella), Chikungunya, Chlamydia Trachomatis, Chronic Wasting Disease, Cholera, Clostridium Difficile Infection, Cryptosporidiosis, Cyclospora Infection, Dengue Fever, Diphtheria, Eastern Equine Encephalitis, Ebola, Ehrlichiosis, E. Coli (STEC), Encephalitis, Arboviral, Enterovirus, Foodborne and Waterborne Diseases, Giardiasis, Gonococcal Disease, Haemophilus Influenzae, Hantavirus Disease, Healthcare Associated Infections, Healthcare Associated Infection Events, Hemolytic Uremic Syndrome, Hepatitis, Hepatitis A, Hepatitis B, Hepatitis C, Hepatitis Delta, Hepatitis E, Herpes, Genital, Herpes Gladiatorum, HIV, Influenza (Seasonal & H1 N1), La Crosse Encephalitis, Legionellosis, Leptospirosis, Listeriosis, Lyme Disease, Malaria, Measles, Meningitis, Meningitis, Viral or Aseptic, Meningococcal Disease-Invasive, Methicillin Resistant Staphylococcus Aureus (MRSA), Middle East Respiratory Syndrome (MERS), Monkeypox, Mosquito-borne Illness, Multi-drug Resistant Organism (MDRO) Resources for Healthcare Facilities, Mumps, Norovirus, ORV (Oral Rabies Vaccination, Pandemic Influenza, Pertussis (Whooping Cough), Plague, Powassan Virus Disease, Pneumococcal Disease, Poliomyelitis, Psittacosis, Q Fever, Rabies, Rash Illness, Rheumatic Fever, Rocky Mountain Spotted Fever, Rubella Congenital Syndrome, Rubella (German Measles), Rubeola (Measles), Ricin, Salmonellosis (Except Typhoid), Scabies, Severe Acute Respiratory Syndrome (SARS), SARS-coronavirus 2 (SARS-CoV2), Shiga Toxin-Producing E. coli (STEC), Shigellosis, Smallpox, St. Louis Encephalitis, Staphylococcus Aureus Infections, Streptococcal Disease, Group A Invasive or Streptococcal TSS, Streptococcal TSS or Streptococcal Disease, Group A, Streptococcal Disease, Invasive Group B, Streptococcal Pneumoniae, Surgical Site Infection, Swine Origin Influenza, Syphilis, Tetanus, Toxic Shock Syndrome, Trichinosis, Tuberculosis, Tularemia, Typhoid Fever, Vancomycin-Resistant Entercoccus, Vancomycin Resistant Staphylococcus aureus or Vancomycin Resistant Intermediate Resistance, Varicella (Chicken Pox), Vibriosis, Viral Hemorrhagic Fever, West Nile Virus, Western Equine Encephalitis, Yellow Fever or Yersinia Enterocolitica.

The DJ-1 inhibitors as taught herein can be used to treat or prevent cancer in an elderly subject and/or a subject otherwise suffering from immunoaging, e.g. a subject having been selected (e.g. diagnosed) to have or having a premature aging disease. The cancer can be selected from the group consisting of adrenal cancer, anal cancer, bile duct cancer, bladder cancer, bone cancer, brain and spinal cord tumors, breast cancer, Castleman disease, cervical cancer, colon cancer, endometrial cancer, esophagus cancer, Ewing family of tumors, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, lymphoma, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, rectum cancer, retinoblastoma, rhabdomyosarcoma, salivary gland cancer, sarcoma, skin cancer, basal and squamous cell cancer, melanoma, merkel cell cancer, small intestine cancer, stomach cancer, testicular cancer, thymus cancer, thyroid cancer, uterine sarcoma, vaginal cancer, vulvar cancer, Waldenstrom macroglobulinemia, or Wilms tumor.

In a further aspect the DJ-1 inhibitors as taught herein can be used as a check point inhibitor. Indeed, as DJ-1 depletion can significantly decrease PD-1 expression among CD4 and CD8 T cells in old mice, DJ-1 inhibitors can be used as an immune checkpoint inhibitor. Immune checkpoint inhibitors are of particular interest in the treatment of diseases, such as but not limited to cancer, more particularly in patients which are identified to be likely to be susceptible to the treatment with a check point inhibitors.

In particular embodiments, the invention provides for the use of an inhibitor of DJ-1 in the treatment (or prevention) of cancer, wherein said method comprises testing the patient for biomarkers such PD-1/PDL1, to determine the suitability of said patient to treatment (or prevention) by an inhibitor of DJ-1. More particularly, expression of biomarkers PD-L1 on the cancer cells of said patient and/or of PD-1 on the immune cells of said patient are indicative of the susceptibility of said patient to treatment (or prevention) with an inhibitor of DJ-1. The inventor thus further provides a method for determining the susceptibility of a patient to treatment (or prevention) with a DJ-1 inhibitor, said method comprises determining the expression of PDL-1 on a cancer cell of said patient. Further embodiments of the invention relate to determining the susceptibility of a patient to treatment (or prevention) with a DJ-1 inhibitor, said method comprises determining tumor mutation burden (TMB) in said patient. Methods for determining TMB are known by the skilled person, and included but are not limited to whole exome sequencing, next generation sequencing, etc.

A further aspect of in the invention relates to the use of an inhibitor of DJ-1 in the treatment (or prevention) of advanced cancer (i.e. cancer that is considered unlikely to or have been shown not to be cured or controlled with treatment), such as advanced melanoma or advanced forms of non-small cell lung cancer (NSCLC), head and neck squamous cell cancer (HNSCC), classical Hodgkin Lymphoma (cHL), urothelial cancer, microsatellite instability-high cancers, gastric cancer, primary mediastinal B cell lymphoma, and cervical cancer.

A further aspect of the invention relates to the use of an inhibitor of DJ-1 as described herein in combination therapies for the treatment (or prevention) of cancer, wherein an inhibitor of DJ-1 as described herein is used as a checkpoint inhibitor in the combination with other types of treatment (or prevention), such as immunotherapies, chemotherapy, radiotherapy, and targeted therapy. In further embodiments the invention provides combination therapies of an inhibitor of DJ-1 as described herein with another therapeutic which is not a PD-1/PD-L1 inhibitor.

As used herein, a phrase such as “a subject in need of treatment” includes subjects that would benefit from treatment of a given condition, particularly a premature aging disease, immunoaging-related disease, or vaccination inefficiency. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed immunoaging-related disease, as well as prophylactic or preventive measures, wherein the aim is to prevent or lessen the chances of incidence of an undesired affliction, such as to prevent occurrence, development and progression of a premature aging disease, immunoaging-related disease, or vaccination inefficiency. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician.

The products and methods as taught herein allow to administer a therapeutically effective amount of an agent as taught herein, such as a DJ-1 inhibitor, in elderly subjects or subjects otherwise suffering from immunoaging, e.g. subjects having been selected (e.g. diagnosed) to have or having a premature aging disease which will benefit from such treatment. The term “therapeutically effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated. Methods are known in the art for determining therapeutically effective doses of an agent as taught herein, such as a DJ-1 inhibitor.

The term “therapeutically effective dose” as used herein refers to an amount of an agent as taught herein, such as a DJ-1 inhibitor, that when administered brings about a positive therapeutic response with respect to treatment of an elderly subject or a subject otherwise suffering from immunoaging, e.g. a patient having been selected (e.g. diagnosed) to have or having a premature aging disease.

Appropriate therapeutically effective doses of an agent as taught herein, such as a DJ-1 inhibitor, may be determined by a qualified physician with due regard to the nature of the immune checkpoint inhibitor, the disease condition and severity, and the age, size and condition of the patient.

Without limitation, a typical dose of a DJ-1 inhibitor to be administered may range from about 1 mg to about 2000 mg, or from about 1 mg to about 1000 mg per administration, for example, from about 5 mg to about 1500 mg or from about 10 mg to about 500 mg per administration. Exemplary doses may include about 1 mg, about 5 mg, about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, or about 500 mg per administration.

For instance, a DJ-1 inhibitor may be administered, optionally in combination with another agent at between about 100 and about 2000 mg/m² body surface per day, such as between about 200 and about 1500 mg/m² body surface per day, such as between about 300 and about 1000 mg/m² body surface per day.

The present inventors found that DJ-1 deficiency resulted in one or more of decreasing the genesis of natural Treg cells, decreasing the number of memory T cells, increasing the number of naïve T cells, decreasing T cell exhaustion, thereby increasing antigen-specific responses. Accordingly, the DJ-1 inhibitor can advantageously be used to boost vaccine efficiency in subjects suffering from or at risk of immunoaging, such as elderly people, which may show diminished vaccine efficacy.

Hence, in a further aspect, the invention provides the use of the inhibitor of DJ-1 as taught herein as an adjuvant, e.g. in a vaccine, in particular in a cancer vaccine or SARS-CoV2 vaccine, preferably a cancer vaccine. For instance, the invention provides the use of the inhibitor of DJ-1 as taught herein as an adjuvant for vaccination of a subject, in particular of an elderly subject, a subject having been selected (e.g. diagnosed) to have or having a premature aging disease, a subject suffering from immunoaging or a subject at risk of immunoaging, such as a patient with accumulated exposure to infectious agents, autoantigens and/or cancer antigens, or a patient having suffered from malnutrition.

The term “vaccination” refers to the administration of a vaccine to help the immune system develop protection from a disease. Vaccines may contain a microorganism or virus in a weakened or killed state, or proteins or toxins from the organism. In stimulating the body's adaptive immunity, vaccines help prevent sickness from an infectious disease.

The DJ-1 inhibitor for administration in combination with an immune response-inducing compound or composition may be administered before, concomitantly with, or after administration of said immune response-inducing compound or composition. In some embodiments, the term “adjuvant” refers to a compound that when administered in conjunction with or as part of an immunogenic composition as described herein augments, enhances and/or boosts the immune response to an immune response-inducing compound or composition, but when the compound is administered alone does not generate an immune response to the immune response-inducing compound or composition. In some embodiments, the adjuvant ensures an increased immune response to the immune response-inducing compound or composition and does not produce an allergy or other adverse reaction.

In certain embodiments, the DJ-1 inhibitor augments the intrinsic response of a subject to a compound or composition capable of inducing an immune response.

The recitation “an immune response-inducing compound or composition” refers to a compound or composition capable of inducing an immune response.

In certain embodiments, the immune response-inducing compound or composition is an antibody response-inducing compound or composition. In certain embodiments, the immune response-inducing compound or composition is an antigen, such as a viral, bacterial, parasitic or other protein subunit; a live, attenuated or inactivated virus; a cell (e.g. dendritic cell); a recombinant vector; or DNA.

A further aspect provides an immunogenic composition comprising an inhibitor of DJ-1 as defined herein and a compound or composition capable of inducing an immune response (or an immune-response inducing compound or composition).

The terms “immunogenic composition” or “vaccine” may be used interchangeably herein and refer to a biological preparation that provides active acquired immunity to a particular disease.

The immunogenic composition may contain an antigen, such as a viral, bacterial, parasitic or other protein subunit. The immunogenic composition may contain a live immune response-inducing compound or composition, such as a live virus. Alternatively or in addition, the immunogenic composition (e.g. vaccine) may contain an inactivated immune response-inducing compound or composition, such as an attenuated or inactivated virus. The immunogenic composition may contain a cell, a recombinant vector, or DNA.

Techniques known to one of skill in the art may be used to inactivate viruses. Common methods use formalin, heat, or detergent for inactivation, e.g., U.S. Pat. No. 6,635,246, which is herein incorporated by reference in its entirety. Other methods include those described in U.S. Pat. Nos. 5,891,705; 5,106,619 and 4,693,981, which are incorporated herein by reference in their entireties.

In certain embodiments, the immunogenic composition may comprise, or may be administered in combination with, a further adjuvant. Specific examples of well-known adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). In specific embodiments, the adjuvant is AS03 (GlaxoSmithKline). In specific embodiments, the adjuvant is MF59 (Novartis). In certain embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomelic amino acids such as polyglutamic acid or polylysine. It should be understood that different immunogenic compositions may comprise different adjuvants or may comprise the same adjuvant.

In certain embodiments, the immunogenic composition may be a cancer vaccine. In certain embodiments, the immunogenic composition may be a therapeutic cancer vaccine. In certain embodiments, the immunogenic composition may be used to reduce Treg frequency or numbers to treat cancer as a therapy. In certain embodiment, the immunogenic composition may be used to reduce IL10-producing cells among CD4 T cells to treat cancer as a therapy.

The term “cancer vaccine” refers to a vaccine that treats existing cancer or prevents development of a cancer. Vaccines that treat existing cancer are known as therapeutic cancer vaccines.

In certain embodiments, the immunogenic composition, in particular the cancer vaccine, may comprise an inhibitor of DJ-1 as defined herein and a tumor-specific antigen.

The term “tumor antigen” refers to an antigenic substance produced in or on tumor cells.

As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

In certain embodiments, the tumor, including any metastases of the tumor, may be of epithelial origin. In certain embodiments, the tumor, including any metastases of the tumor, may originate from glial cells, astrocytes, oligodendrocyte progenitor cells or neural stem cells.

Tumors of epithelial origin include any tumors originated from epithelial tissue in any of several sites, such as without limitation breast, lung, bladder, cervix, intestine, colon, skin, head and neck (including lips, oral cavity, salivary glands, nasal cavity, nasopharynx, paranasal sinuses, pharynx, throat, larynx, and associated structures), esophagus, thyroid, kidney, liver, pancreas, penis, testes, prostate, vagina, or anus.

In certain embodiments, the tumor antigen may be a shared tumor antigen (i.e., an antigen expressed by many tumors). In certain embodiments, the tumor antigen may be an unique tumor antigen (i.e., an antigen expressed only by individual tumors). Unique tumor antigen often derive from mutation of genes that have relevant biological functions.

In certain embodiments, the immunogenic composition, in particular the cancer vaccine, may comprise an inhibitor of DJ-1 as defined herein and whole tumor cells.

In certain embodiments of the products or the methods as taught herein, the cancer vaccine may be allogeneic to the subject to be treated. The terms “allogeneic” or “homologous” with reference to the cancer vaccine denotes that the cancer vaccine is obtained from one or more (pooled) subjects other than the subject to be contacted or treated with the cancer vaccine.

In certain embodiments of the products or the methods as taught herein, the cancer vaccine may be autologous to the subject to be treated. The term “autologous” with reference to the cancer vaccine denotes that the cancer vaccine is obtained from the same subject to be contacted or treated with the cancer vaccine.

In certain embodiments of the products or the methods as taught herein, the cancer vaccine may comprise a mixture of autologous and allogeneic (i.e., homologous) cancer vaccines as defined above.

In certain embodiments, the immunogenic composition is a SARS-CoV2 vaccine.

Also provided in an aspect is an immunogenic composition, in particular a cancer vaccine or SARS-CoV2 vaccine, preferably a cancer vaccine, comprising an inhibitor of DJ-1.

A further related aspect provides a kit of parts comprising an inhibitor of DJ-1 as defined herein and a compound or composition capable of inducing an immune response, e.g. a compound or composition capable of inducing an antibody response.

The terms and recitations “kit”, “kit of parts” or “assembly” can be used interchangeably herein.

Both the DJ-1 inhibitor and the immune response-inducing compound or composition can be considered active components. The DJ-1 inhibitor can advantageously increase the induction of an antigen-specific immune response of a subject against the immune response-inducing compound or composition.

The terms “active ingredient” or “active component” can be used interchangeably and broadly refer to a compound or substance which, when provided in an effective amount, achieves a desired outcome. The desired outcome may be therapeutic and/or prophylactic. Typically, an active ingredient may achieve such outcome(s) through interacting with and/or modulating living cells or organisms.

The term “active” in the recitations “active ingredient” or “active component” refers to “pharmacologically active” and/or “physically active”.

The reference to “an active ingredient” encompasses one or more of the active ingredient, such as two or more, three or more, or four or more, such as five, six, seven, eight or more of the active ingredient. For instance, the reference to a DJ-1 inhibitor encompasses one or more DJ-1 inhibitors, such as two or more, three or more, or four or more, such as five, six, seven, eight or more DJ-1 inhibitors.

Certain aspects provided herein relate to:
a pharmaceutical composition comprising an inhibitor of DJ-1 as taught herein and a pharmaceutically acceptable carrier, for use in treating or preventing immunoaging in a subject.
a pharmaceutical composition comprising an inhibitor of DJ-1 as taught herein and a pharmaceutically acceptable carrier, for use in treating or preventing a premature aging disease in a subject, in particular in a subject having been selected (e.g. diagnosed) to have or having a premature aging disease.
a pharmaceutical composition comprising an inhibitor of DJ-1 as taught herein and a pharmaceutically acceptable carrier, for use in rejuvenating the immune system in a subject, in particular in an elderly subject or in a subject having been selected (e.g. diagnosed) to have or having a premature aging disease.
a pharmaceutical composition comprising an inhibitor of DJ-1 as taught herein and a pharmaceutically acceptable carrier, for use in treating or preventing an immunoaging-related disease in an elderly subject or in a subject having been selected (e.g. diagnosed) to have or having a premature aging disease.
a pharmaceutical composition comprising an inhibitor of DJ-1 as taught herein and a pharmaceutically acceptable carrier, for use in treating or preventing vaccination inefficiency in a subject, in particular in an elderly subject or in a subject having been selected (e.g. diagnosed) to have or having a premature aging disease.

The terms “pharmaceutical formulation”, pharmaceutical composition”, “formulation” or “composition” may be used interchangeably herein.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical or immunogenic compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical or immunogenic compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

For example, for oral administration, pharmaceutical or immunogenic compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical or immunogenic composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical or immunogenic compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilizing or dispersing agents known in the art. Suitable pharmaceutical or immunogenic formulations for administration in the form of aerosols or sprays are, for example, solutions, suspensions or emulsions of the agents as taught herein or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical or immunogenic compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The agents and pharmaceutically acceptable salts thereof of the invention can also be lyophilized and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

Suitable carriers for microcapsules, implants or rods are, for example, copolymers of glycolic acid and lactic acid.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The dosage or amount of the present active agents used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) as taught herein.

Without limitation, depending on the type and severity of the disease, a typical daily dosage of an agent as disclosed herein, or combinations of two or more such agents, might range from about 1 μg/kg to 1 g/kg of body weight or more, depending on the factors mentioned above. For instance, a daily dosage of the agent(s) may range from about 1 mg/kg to 1 g/kg of body weight. For repeated administrations over several days or longer, depending on the condition, the treatment is sustained until a desired suppression of disease symptoms occurs. A preferred dosage of the agent(s) may be in the range from about 10.0 mg/kg to about 500 mg/kg of body weight. Thus, one or more doses of about 10.0 mg/kg, 20.0 mg/kg, 50.0 mg/kg, 100 mg/kg, 200 mg/kg, 300 mg/kg, 400 mg/kg, or 500 mg/kg (or any combination thereof) may be administered to the patient. Such doses may be administered intermittently, e.g., every day, every week or every two or three weeks.

In certain embodiments, the agent(s) may be administered daily during the treatment. In certain embodiments, the agent(s) may be administered at least once a day during the treatment, for example the agent(s) may be administered at least twice a day during the treatment, for example the agent(s) may be administered at least three times a day during the treatment. In certain embodiments, the agent(s) may be administered continuously during the treatment for instance in an aqueous drinking solution.

In certain embodiments, the DJ-1 inhibitor or pharmaceutical formulation or immunogenic formulation as taught herein may be used alone or in combination with one or more active compounds that are suitable in the treatment of diseases as disclosed herein. The latter can be administered before, after, or simultaneously with the administration of the DJ-1 inhibitor or pharmaceutical formulation or immunogenic formulation as taught herein.

Certain embodiments provide an inhibitor of DJ-1, an immunogenic composition, or a kit of parts, as taught herein, for use in treating or preventing vaccination inefficiency in a subject, in particular in an elderly subject or in a subject having been selected (e.g. diagnosed) to have or having a premature aging disease.

An aspect thus provides an inhibitor of DJ-1, an immunogenic composition, or a kit of parts, as taught herein, for use in treating or preventing vaccination inefficiency in a subject, in particular in an elderly subject or in a subject having been selected (e.g. diagnosed) to have or having a premature aging disease.

In certain embodiments, the subject has been selected to have or has a premature aging disease; or the subject is an elderly subject, preferably wherein the subject has an age of at least 60 years, more preferably of at least 65 years.

Related embodiments or aspects provide:
a method of treating (or preventing) vaccination inefficiency in a subject, in particular in an elderly subject or in a subject having been selected (e.g. diagnosed) to have or having a premature aging disease, comprising administering to the subject a therapeutically or prophylactically effective amount of an inhibitor of DJ-1, an immunogenic composition, or a kit of parts, as taught herein.
the use of an inhibitor of DJ-1, an immunogenic composition, or a kit of parts, as taught herein, for the manufacture of a medicament for use in treatment (or prevention) of vaccination inefficiency.
the use of an inhibitor of DJ-1, an immunogenic composition, or a kit of parts, as taught herein, for the treatment (or prevention) of vaccination inefficiency.

Certain embodiments or aspects provide:
an inhibitor of DJ-1, an immunogenic composition, or a kit of parts, as taught herein, for use in increasing vaccination inefficiency in a subject.
an inhibitor of DJ-1, an immunogenic composition, or a kit of parts, as taught herein, for use in increasing an immune response to an infectious disease or cancer in a subject.

In elderly people or in subjects having been selected (e.g. diagnosed) to have or having a premature aging disease, the vaccine efficiency may be reduced.

The phrases “vaccination inefficiency” or “vaccine inefficiency” as used herein refers to a vaccination efficiency which is reduced relative to (i.e., as compared to) the vaccination efficiency of a control sample. The control sample may be a sample of a healthy subject or of a group (e.g. population) of healthy subjects, such as age- and/or gender-matched healthy subject(s) or healthy (gender-matched) subject(s) at an earlier age.

The terms “vaccination efficiency” or “vaccine efficiency” may be used interchangeably herein.

In certain embodiments, the method of treating (or preventing) vaccination inefficiency comprises a step of diagnosing a subject with a premature aging disease comprising determining the presence of one or more of the criteria (e.g. markers) of immunoaging as described elsewhere herein.

As used herein, the vaccination efficiency of a subject may be determined by measuring the percentage of antigen-specific T cells among total CD4 or CD8 T cells following vaccination of the subject. The higher the percentage of antigen-specific T cells among total CD4 or CD8 T cells following vaccination, the higher the vaccination efficiency. The vaccine efficiency may be measured using a tetramer assay (also known as a tetramer stain).

The term “tetramer assay” refers to a procedure that uses tetrameric proteins to detect and quantify T cells that are specific for a given antigen e.g. within a blood sample. In brief, the tetramers used in the assay are made up of four major histocompatibility complex (MHC) molecules, which are found on the surface of most cells in the body. MHC molecules present peptides to T-cells as a way to communicate the presence of viruses, bacteria, cancerous mutations, or other antigens in a cell. If a T-cell's receptor matches the peptide being presented by an MHC molecule, an immune response is triggered. Thus, MHC tetramers that are bioengineered to present a specific peptide can be used to find T-cells with receptors that match that peptide. The tetramers may be labeled with a fluorophore, allowing tetramer-bound T-cells to be analysed with flow cytometry.

In certain embodiments, the vaccination efficiency of a subject having been selected (e.g. diagnosed) to have or having a premature aging disease may be reduced by at least 1% relative to (i.e., compared with) (i.e., the vaccination efficiency of a subject having been selected to have or having a premature aging disease may be at most 0.99-fold) the vaccination efficiency of a healthy age- and/or gender-matched subject, or a group of healthy age- and/or gender-matched subjects. In certain embodiments, the vaccination efficiency of a subject having been selected (e.g. diagnosed) to have or having a premature aging disease may be reduced by at least 5% (i.e. at most 0.95-fold), at least 10% (i.e. at most 0.90-fold), at least 20% (i.e. at most 0.80-fold), at least 30% (i.e. at most 0.70-fold), at least 40% (i.e. at most 0.60-fold), at least 50% (i.e. at most 0.50-fold), at least 60% (i.e. at most 0.40-fold), at least 70% (i.e. at most 0.30-fold), at least 80 (i.e. at most 0.20-fold), at least 90% (i.e. at most 0.10-fold), or at least 95% (i.e. at most 0.05-fold) relative to (i.e., compared with) the vaccination efficiency of a healthy age- and/or gender-matched subject, or a group of healthy age- and/or gender-matched subjects.

In certain embodiments, the vaccination efficiency of an elderly subject may be reduced by at least 1% relative to (i.e., compared with) (i.e., the vaccination efficiency of an elderly subject may be at most 0.99-fold) the vaccination efficiency of a healthy (gender-matched) subject at an earlier age or a group of healthy (gender-matched) subjects at an earlier age, such as a healthy adult (gender-matched) subject or a group of healthy adult (gender-matched) subjects. In certain embodiments, the vaccination efficiency of an elderly subject may be reduced by at least 5% (i.e. at most 0.95-fold), at least 10% (i.e. at most 0.90-fold), at least 20% (i.e. at most 0.80-fold), at least 30% (i.e. at most 0.70-fold), at least 40% (i.e. at most 0.60-fold), at least 50% (i.e. at most 0.50-fold), at least 60% (i.e. at most 0.40-fold), at least 70% (i.e. at most 0.30-fold), at least 80 (i.e. at most 0.20-fold), at least 90% (i.e. at most 0.10-fold), or at least 95% (i.e. at most 0.05-fold) relative to (i.e., compared with) the vaccination efficiency of a healthy (gender-matched) subject at an earlier age or a group of healthy (gender-matched) subjects at an earlier age, such as a healthy adult (gender-matched) subject or a group of healthy adult (gender-matched) subjects.

In certain embodiments of the methods or uses, as taught herein, by the administration of the DJ-1 inhibitor, the vaccination efficiency of the subject may increase by at least about 1% (i.e., 1.01-fold), at least about 5% (i.e., 1.05-fold), at least about 10% (i.e., 1.10-fold), at least about 20% (i.e., 1.20-fold), at least about 30% (i.e., 1.30-fold), at least about 40% (i.e., 1.40-fold), at least about 50% (i.e., 1.50-fold), at least about 60% (i.e., 1.60-fold), at least about 70% (i.e., 1.70-fold), at least about 80% (i.e., 1.80-fold), at least about 90% (i.e., 1.90-fold), at least about 95% (i.e., 1.95-fold), or at least about 100% relative to (i.e., compared with) (i.e., 2-fold) the vaccination efficiency of a control sample of a corresponding subject or group of subjects as defined herein. In certain embodiments of the methods or uses, as taught herein, by the administration of the DJ-1 inhibitor, the vaccination efficiency of the subject may be at least about 2-fold, at least about 5-fold, at least about 10-fold, at least about 20-fold, at least about 30-fold, at least about 40-fold, or at least about 50-fold, the vaccination efficiency of a control sample of a corresponding subject or group of subjects as defined herein. Such increased vaccination efficiency advantageously allows a subject suffering from immunoaging to have increased immune responses, e.g. against infectious diseases.

In certain aspects or embodiments, provided herein is an inhibitor of DJ-1, an immunogenic composition, or a kit of parts, as taught herein, for use in a method of immunizing a subject against an infectious disease or cancer, wherein the method comprises administering to the subject the DJ-1 inhibitor, immunogenic composition or kit of parts, as taught herein.

In certain aspects or embodiments, provided herein is a kit of parts as taught herein, for use in a method of immunizing a subject against an infectious disease or cancer, wherein the method comprises (i) a first administration of the DJ-1 inhibitor as defined herein to the subject; and (ii) a second administration of an immune response-inducing compound or composition to the subject. In certain embodiments, the first and second administrations may be separated by at least 30 minutes, at least 1 hour, at least 2 hours, at least 4 hours, at least 6 hours, at least 8 hours, at least 12 hours, at least 24 hours (i.e., at least 1 day), at least 2 days, at least 3 days, at least 5 days, at least 10 days, at least 15 days, at least 30 days, at least 2 months, or at least 6 months. In specific embodiments, the first and second administrations may be separated by 1 hour to 6 hours, 2 hours to 12 hours, 4 hours to 8 hours, 6 hours to 24 hours, 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months.

The present application also provides aspects and embodiments as set forth in the following Statements:

Statement 1. An inhibitor of DJ-1 (PARK7) for use in treating or preventing immunoaging in a subject.

Statement 2. The inhibitor for use according to statement 1, wherein the inhibitor of DJ-1 binds to DJ-1 or a polynucleotide encoding DJ-1, preferably wherein the inhibitor of DJ-1 specifically binds to DJ-1 or a polynucleotide encoding DJ-1.

Statement 3. The inhibitor for use according to statement 1 or 2, wherein the inhibitor is one or more agents selected from the group consisting of a chemical substance, an antibody, an antibody fragment, an antibody-like protein scaffold, a protein or polypeptide, a peptide, a peptidomimetic, an aptamer, a photoaptamer, a spiegelmer, a nucleic acid, a gene-editing system, an antisense agent, an RNAi agent, and a soluble receptor.

Statement 4. The inhibitor for use according to statement 3, wherein the chemical substance is an organic molecule, preferably a small organic molecule, or wherein the nucleic acid is an oligonucleotide.

Statement 5. The inhibitor for use according to statement 3, wherein the inhibitor is one or more agents selected from the group consisting of an antibody, an antibody fragment, an antibody-like protein scaffold, a nucleic acid, a gene-editing system, an antisense agent, and an RNAi agent.

Statement 6. The inhibitor for use according to any one of statements 3 to 5, wherein the RNAi agent has a sequence identity of at least 50%, 60%, 70%, 80%, 90%, 95%, 98%, 99% or 100% to SEQ ID NO: 4.

Statement 7. The inhibitor for use according to any one of statements 3 to 5, wherein the nucleic acid or the RNAi agent is provided within a plasmid vector and/or wherein the nucleic acid or the RNAi agent is modified or encapsulated by synthetic or natural nanoparticles; preferably wherein the nanoparticle is a liposomal nanoparticle.

Statement 8. The inhibitor for use according to any one of statements 1 to 7, wherein the subject is a human subject.

Statement 9. The inhibitor for use according to any one of statements 1 to 8, wherein the subject has been selected to have or has a premature aging disease; or wherein the subject is an elderly subject, preferably wherein the subject has an age of at least 60 years, more preferably of at least 65 years.

Statement 10. The inhibitor of DJ-1 for use according to any one of statements 1 to 8, for use in treating or preventing an immunoaging-related disease in a subject, wherein the subject has been selected to have or has a premature aging disease; or wherein the subject is an elderly subject.

Statement 11. The inhibitor for use according to statement 10, wherein the immunoaging-related disease is cancer or an infectious disease.

Statement 12. The inhibitor for use according to any one of statements 1 to 8, for use in treating or preventing a premature aging disease in a subject.

Statement 13. The inhibitor for use according to any one of statements 9 to 12, wherein the premature aging disease is selected from the group consisting of Hutchinson-Gilford progeria syndrome (HGPS), Bloom syndrome (BS), Cockayne syndrome (CS), Mandibuloacral Dysplasia with Type A Lipodystrophy (MADA); Werner syndrome (WS), Rothmund-Thomson syndrome (RTS), Seip syndrome, xeroderma pigmentosum (XP), trichothiodystrophy (TTD), combined xeroderma pigmentosum-Cockayne syndrome (XP-CS), and restrictive dermopathy (RD); preferably wherein the premature aging disease is Hutchinson-Gilford progeria syndrome.

Statement 14. The inhibitor for use according to statement 11 or 12, wherein the subject has been selected to have or has a premature aging disease.

Statement 15. Use of an inhibitor of DJ-1 as defined in any one of statements 1 to 7, as an adjuvant.

Statement 16. An immunogenic composition comprising an inhibitor of DJ-1 as defined in any one of statements 1 to 7 and a compound or composition capable of inducing an immune response.

Statement 17. The immunogenic composition according to statement 16, wherein the immunogenic composition is a cancer vaccine.

Statement 18. A kit of parts comprising an inhibitor of DJ-1 as defined in any one of statements 1 to 7 and a compound or composition capable of inducing an immune response.

Statement 19. The inhibitor of DJ-1 as defined in any one of statements 1 to 7, an immunogenic composition as defined in statement 16 or 17, or a kit of parts as defined in statement 18, for use in treating or preventing vaccination inefficiency in a subject.

Statement 20. The inhibitor of DJ-1 for use according to statement 19, the immunogenic composition for use according to statement 19, or the kit of parts for use according to statement 19, wherein the subject has been selected to have or has a premature aging disease; or wherein the subject is an elderly subject, preferably wherein the subject has an age of at least 60 years, more preferably of at least 65 years.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

Sequence Listing

Throughout the description and Examples, reference is made to the following sequences:
SEQ ID NO: 1: human DJ-1 mRNA sequence annotated under NCBI Reference Sequence NM_007262.5
SEQ ID NO: 2: human DJ-1 mRNA sequence annotated under NCBI Reference Sequence NM_001123377.1
SEQ ID NO: 3: human DJ-1 protein sequence annotated under Uniprot accession number Q99497
SEQ ID NO: 4: exemplary anti-DJ-1 siRNA

### EXAMPLES

Materials and Methods

Human-Related Experiments

Human T Cell Isolation

The buffy coats or leukopaks from healthy donors used for primary T cell culture and analysis were provided by the Luxembourg Red Cross. Human primary natural Tregs and Teffs (CD4 effector T cells) were derived respectively from sorted CD4+CD25highCD127low and CD4+CD25− T cells of peripheral blood mononuclear cells (PBMC) on BD FACSAria™ III cell sorter. The PBMCs were isolated by using Ficol-Paque plus (17-1440-03, GE Healthcare) or Lympoprep (07801, StemCell) followed by magnetic separation with anti-human CD4 microbeads (130-045-101, MACS Miltenyi Biotec), as described by the manufacturers, before being stained with mouse monoclonal [RPA-T4] anti-human CD4 FITC (555346, BD Biosciences) (dilution 1:20), mouse monoclonal [M-A251] anti-human CD25 APC (555434, BD Biosciences) (dilution 1:20) and mouse monoclonal [HIL-7R-M21] anti-human CD127 V450 (560823, BD Biosciences) (dilution 1:20) for sorting with BD FACSAria™ III.

We complied with all the relevant ethic regulations and Luxembourg CNER (Comité National d'Ethique de Recherche) has approved the PD patients related study. The family carrying the c.192G>C mutation in the DJ-1 gene has been initially described elsewhere (Hering et al., 2004, Hum Mutat 24, 321-329) and written informed consent for all participating individuals was obtained. The index patient (56 years) carries the homozygous c.192G>C mutation and has been affected by PD for 22 years. The general disease progression over time is benign with a retained good response to levodopa therapy. The patient was currently presenting with a bilateral akinetic rigid syndrome more pronounced on the left side with some postural instability and variable gait problems not interfering with his autonomy and corresponding to a Hoehn&Yahr stage 3. The two unaffected siblings (60 and 63 years) are both heterozygous carriers of the c.192G>C mutation and were devoid of any clinical sign of PD at the recent neurological examination, as expected for this autosomal-recessively inherited condition. Before analysis, the experimenters were blinded to the patients' genotypes. PBMC isolation was performed by using SepMate tubes and lymphoprep systems (StemCell, 86450 and 07801, respectively).

Human T Cell Culturing

We followed the same protocol as described in our previous work (He et al., 2012, Molecular systems biology 8, 624). Sorted T cells were cultured in IMDM (21980-032, Thermo Fisher Scientific) complete medium supplemented with 10% heat-inactivated (56° C., 45 min) Gibco® fetal bovine serum (FBS) (10500-064, Thermo Fisher Scientific), 1× Penicillin+Streptomycin (15070-063, Thermo Fisher Scientific), 1×MEM non-essential amino acids (M7145, Sigma-Aldrich) and 50 μM beta-mercaptoethanol (M7522, Sigma-Aldrich). All the human T cells were cultured in 37° C. 7.5% CO2 incubators, unless specified. 100 U/ml recombinant human IL2 (known as Proleukin® in medication) (PZN 2238131, Novartis) was added daily to Treg (but not Teff) cell culture medium and the same amount of IL2 was added to Tregs unless otherwise stated. Every seven days, all T-cells were restimulated with irradiated Epstein-Barr virus (EBV preparation from VR-1492, ATCC)-transformed B-cells3 (EBV-B cells), at a 1:1 ratio of T and EBV-B cells, to expand and maintain the T-cells. The EBV-B cells were irradiated in an RS2000 X-Ray Biological Irradiator (Rad Source Technologies) for 30 min at a rate of 2.80 Gy/min. The T-cells were regularly characterized by co-staining CD4, CD25, FOXP3 and Helios protein levels by flow cytometry. When the primary Tregs were older than 6 weeks or the expression level of FOXP3 or Helios or the cellular viability was apparently decreased, the cells were discarded and new T cells were isolated from different healthy donors. The antibodies used for cell quality assessment (CQA) were: mouse monoclonal [RPA-T4] anti-human CD4 BUV395 (564724, BD Biosciences) (dilution 1:100), mouse monoclonal [M-A251] anti-human CD25 FITC (555431, BD Biosciences) (dilution 1:100), mouse monoclonal [22F6] anti-human Helios Pacific blue (13721, BioLegend) (dilution 1:100), mouse monoclonal [206D] anti-human FOXP3 Alexa Fluor 647 (320119, BioLegend) (dilution 1:10). The LIVE/DEAD® Fixable Near-IR Dead Cell Stain (L10119, Thermo Fisher Scientific), diluted 1:500, was used to distinguish living cells from dead cells. In certain cases, we directly compared the markers of our isolated human Tregs with TregThu, a golden standard isolated from our previous work. The procedure for the staining of extracellular and intracellular markers is described below.

Gene Knockdown

Targeted gene expression was knocked-down by using P3 Primary Cell 4D-Nucleofector X Kit L (V4XP-3024, Lonza) 90 with 100 μl P3 primary cell solution and 100 pmol of the corresponding si_RNAs (resuspended in 10 μl RNAse-free H₂O): si_Non-Specific (si_NS or si_CTL) (sc-37007, Santa Cruz), si_FOXP3 (SI04955062, Qiagen) and si_PARK7/_DJ-1 (SI00301091, Qiagen). Amaxa 4D-Nucleofector™ X System (Lonza) was used for the experiments with the manufacturer's recommended program for stimulated human primary T cells. In order to ensure a similar knockdown efficiency between single and double knockdown, we used the same number of T cells for different conditions and normalized the amount of total RNA in the single knockdown with an additional 10 μl si-NS. Following the siRNA transfection procedure, T cells were transferred into 12-well plates with pre-warmed complete medium supplemented with 100 U/ml IL-2 for Tregs and kept at 37° C. for 1 day. They were then stimulated with Dynabeads Human T-Activator CD3/CD28 (11131D, Thermo Fisher Scientific) (ratio of cells and beads: 1:1) or with ImmunoCult Human T Cell Activator (10991, STEMCELL Technologies) (25 μl/ml) in 24-well plates for 1 or 2 days depending on the corresponding experiments, with or without additional recombinant IL-2 for Tregs and Teffs, respectively.

RNA Extraction and cDNA Synthesis

RNA was extracted as previously described (He et al., 2012, Molecular systems biology 8, 624). RNA samples for standard and quantitative PCR were prepared by using the RNeasy Mini Kit (74106, Qiagen) starting with lysing the cells with RLT buffer supplemented with 1% beta-mercaptoethanol (63689, Sigma-Aldrich), following the manufacturer's instructions and including the digestion of genomic DNA with DNAse I (79254, Qiagen). The RNA concentration was measured with a NanoDrop 2000c Spectrophotometer (Thermo Fisher Scientific) followed by a quality check of the RNA integrity number (RIN). For RIN assessment, the Agilent RNA 6000 Nano kit (5067-1511, Agilent) was used together with the Agilent 2100 Bioanalyzer Automated Analysis System (Agilent) according to the manufacturer's protocol.

A maximum of 500 ng RNA was used for cDNA synthesis. A master mix for the first step was prepared with 0.5 μl of 50 μM Oligo(dT)20 primers (18418020, Thermo Fisher Scientific), 0.5 μl of 0.09 units/μl Random Primers (48190-011, Thermo Fisher Scientific), 1 μl of 10 mM dNTP mix (R0192, Thermo Fisher Scientific) and RNAse-free water made up to a final volume of 13 μl in 0.2 ml PCR Tube Strips (732-0098, Eppendorf). The tubes were transferred into a Professional Standard Gradient 96 Thermocycler (Biometra) for 5 min at 65° C. and 2 min at 4° C. After the first step, the reaction was supplemented with 40 units RNaseOUT™ Recombinant Ribonuclease Inhibitor (10777019, Thermo Fisher Scientific), 200 units SuperScript™ III Reverse Transcriptase (18080-044, Thermo Fisher Scientific) and dithiothreitol (DTT) (70726, Thermo Fisher Scientific) to give a final concentration of 5 mM in a total reaction volume of 20 μl. The PCR tubes were returned to the thermocycler at 50° C. for 60 min, 70° C. for 15 min and 4° C. until further usage.

Standard Polymerase Chain Reaction (ST-PCR) and Realtime Quantitative PCR (qPCR)

The DreamTaq Green PCR Master Mix (K1081, Thermo Fisher Scientific) was used as a base for the ST-PCR. Forward and reverse primers together with cDNA and RNAse-free water were added. PCR was performed in a Professional Standard Gradient 96 Thermocycler (Biometra). Following the amplification, the samples and MassRuler DNA Ladder Mix (SM0403, Thermo Fisher Scientific) were loaded onto 2% agarose (A9539, Sigma-Aldrich) gel with ethidium bromide (E1510, Sigma-Aldrich) or SYBR Safe (S33102, Thermo Fisher Scientific, dilution 1:10 000) and the gel was run for 1 hr at 120 V in TAE buffer. Images of the bands were taken with the G:Box gel doc system (Syngene).

Real-time quantitative PCR (RT-PCR/qPCR) was performed by using LightCycler 480 SYBR Green I Master Mix (04707516001, Roche), supplemented with cDNA and primers in a reaction volume of 10 μl. LightCycler 480 Multi-well White Plates (04729749 001, Roche) with 384 wells and LC 480 Sealing Foil (04729757001, Roche) were used in these experiments. The reaction was undertaken on a LightCycler 480 (384) platform (LightCycler 480 (384), Roche). The results were analysed with LightCycler 480 SW 1.5 software. The annealing temperature of 55° C. was set for the various genes unless stated.

The primers used for qPCR were: RPS9 (QT00233989, Qiagen) or GAPDH (QT00079247, Qiagen) as reference genes, CSF2 (QT00000896, Qiagen), CTLA4 (QT01670550, Qiagen), FOXP3 (QT00048286, Qiagen), GATA3 (QT00095501, Qiagen), IFNG (QT00000525, Qiagen), IKZF2 (QT00058758, Qiagen), IKZF4 (QT00061138, Qiagen), IL-4 (QT00012565, Qiagen), IL-5 (QT00001435, Qiagen), IL-13 (QT00000511, Qiagen), LGALS3 (QT01026725, Qiagen), PLAU (QT00013426, Qiagen), S1PR1 (QT00208733, Qiagen), PARK7 (QT00055811, Qiagen), TGFB1 (QT00000728, Qiagen), TNF (QT00029162, Qiagen), TNFRSF18 (QT00210728, Qiagen), IL-2 (forward: GTC ACA AAC AGT GCA CCT AC, reverse: ATG GTT GCT GTC TCA TCA GC, Eurogentec), GARP (forward: GAT GGG GAA ACT GAG GCT TAG GAA, reverse: ACC CCC AAT CTC ACC CCA CAA ATA, Eurogentec), LGMN (forward: CTC GCT CCA GGA CCT TCT TCA CAA, reverse: GCT TCC TGC TCC TCA AAA CTA ACA, Eurogentec).

Flow Cytometry (FACS) and ImageStream for Human Cells

Once the cells had been collected and the medium removed, the extracellular proteins of the cells were stained in FACS buffer (PBS (14190169, Thermo Fisher Scientific) containing 2% heat-inactivated FBS (10500-064, Thermo Fisher Scientific) with the cell surface antibodies, the concentration of which was first optimized, for 30 min at 4° C. in the dark. Intracellular staining was performed by using the FOXP3 staining kit (421403, BioLegend) following the manufacturer's instructions. Stained cells were analysed with BD LSRFortessa™. Dead cells were excluded from the analysis by LIVE/DEAD® Fixable Near-IR Dead Cell Stain (L10119, Thermo Fisher Scientific) (dilution 1:500).

Various combinations of the following antibodies were used for FACS analysis: mouse monoclonal [RPA-T4] anti-human CD4 APC (561840, BD Biosciences) (dilution 1:200), mouse monoclonal [RPA-T4] anti-human CD4 FITC (555346, BD Biosciences) (dilution 1:200), mouse monoclonal [G44-26] anti-human CD4 V450 (561292, BD Biosciences) (dilution 1:200), mouse monoclonal [RPA-T4] anti-human CD4 BUV395 (564724, BD Biosciences) (dilution 1:200), mouse monoclonal [L200] anti-human CD4 PE-Cy7 (560644, BD Biosciences) (dilution 1:200), mouse monoclonal [M-A251] anti-human CD25 APC (555434, BD Biosciences) (dilution 1:200), mouse monoclonal [M-A251] anti-human CD25 FITC (555431, BD Biosciences) (dilution 1:200), mouse monoclonal [M-A251] anti-human CD25 V450 (560356, BD Biosciences) (dilution 1:200), mouse monoclonal [HIL-7R-M21] anti-human CD127 V450 (560823, BD Biosciences) (dilution 1:200), mouse monoclonal [50G10] anti-human GARP (221 011, Synaptic Systems GmbH) (dilution 1:100), mouse monoclonal [B56] anti-human Ki-67 V450 (561281, BD Biosciences) (dilution 1:100), mouse monoclonal [206D] anti-human FOXP3 Alexa Fluor 647 (320119, Biolegend) (dilution 1:10), mouse monoclonal IgG1 (50-167-013, BioLegend) (dilution 1:10), mouse monoclonal [22F6] anti-human Helios Pacific blue (137210, BioLegend) (dilution 1:100), rabbit monoclonal [EP2815Y] anti-human PARK7/DJ-1 Alexa Fluor 488 (ab203989, Abcam) (dilution 1:100), rabbit monoclonal [EPR11097(B)] anti-human PDHB Alexa Fluor 594 (ab211838, Abcam) (dilution 1:100), including strict negative controls (rabbit monoclonal [EPR25A] IgG Alexa Fluor 488 (ab199091, Abcam) (dilution 1:50), rabbit monoclonal [EPR25A] IgG Alexa Fluor 594 (ab208568, Abcam) (dilution 1:100), mouse monoclonal [GB12] anti-human GZMB APC (MHGB05, Thermo Fisher Scientific) (dilution 1:200), CellTrace CFSE Cell Proliferation Kit FITC (C34554, Thermo Fisher Scientific) (dilution 1:5000). The FCS files from human-related experiments were analysed by FlowJo 7.6.5 or FlowJo v10 (Tree Star).

For ImageStream experiments, Tregs and Teffs were stimulated and stained as described above, but with staining panels other than those for the FACS analysis. We used the following staining panels: LIVE/DEAD® Fixable Near-IR Dead Cell Stain (L10119, Thermo fisher Scientific) (dilution 1:500), mouse monoclonal [RPA-T4] anti-human CD4 V450 (560346, BD Biosciences) (dilution 1:100), rabbit monoclonal [EP2815Y] anti-human PARK7/DJ-1 Alexa Fluor 488 (ab203989, Abcam) (dilution 1:50), rabbit monoclonal [EPR11097(B)] anti-human PDHB Alexa Fluor 594 (ab211838, Abcam) (dilution 1:100), including strict negative controls (rabbit monoclonal [EPR25A] IgG Alexa Fluor 488 (ab199091, Abcam) (dilution 1:50) and rabbit monoclonal [EPR25A] IgG Alexa Fluor 594 (ab208568, Abcam) (dilution 1:100). The samples were acquired on an ImageStream®X Mark II Imaging Flow Cytometer (Amnis, EMD Millipore) at 60× magnification. The results were analysed by using IDEAS 6.2 software (Amnis) and the Similarity score between PDHB and DJ-1 was determined by means of a similarity dilate algorithm often used for the nuclear translocation quantification of a transcription factor.

We utilized the PDHB-positive area as the targeted location and then calculated to what degree DJ-1 was translocated to the PDHB-positive areas. Only cells gated as living focused singlets were included in the analysis.

TCR Beta Repertoire Sequencing

PBMC isolation from the three patients (P1, P2 and P3) with DJ-1 mutation was performed by using SepMate tubes and Lymphoprep systems (StemCell, 86450 and 07801, respectively). PMBCs were cryopreserved in liquid nitrogen in aliquots of 5×106 cells in 1 ml FBS+10% DMSO. The thawn PBMCs were washed twice with warm (37° C.) supplemented IMDM and recovered over night at 37° C., 7.5% CO2. Cell surface antibodies used to stain and FACS sort naïve and memory CD4 and CD8 T cells from patients' PBMC are listed in Table 1.

TABLE 1
List of antibodies used for patients’ PBMC sorting (TCR repertoire analysis)

| | | | Fluoro- | Catalogue | Dilution |
|---|---|---|---|---|---|
| Antibody | Clone | Company | chrome | number | factor |
| Fc Blocking Abs | / | BD Bioscience | / | 564765 | 1:50 |
| CD3 | HIT3a | BD Bioscience | BV510 | 741822 | 1:100 |
| CD4 | RPA-T4 | BD Bioscience | FITC | 555346 | 1:100 |
| CD8 | RPA-T8 | Biolegend | BV605 | 301040 | 1:100 |
| CD25 | M-A251 | BD Bioscience | APC | 555434 | 1:50 |
| CD45RA | HI100 | Biolegend | Pacific Blue | 304123 | 1:50 |
| CD45RO | UCHL1 | BD Bioscience | PE-CF594 | 562299 | 1:50 |

Genomic DNA (gDNA) was extracted from the sorted naïve and memory CD4 and CD8 T cells using the QIAamp DNA Blood Mini Kit (Qiagen, 51104) and following the manufacture's instructions. The gDNA was eluted in 55ul RNase- and DNase-free water to match the volume and cencentration requirements for survey analysis of TCR beta repertoire sequencing by ImmunoSEQ (Adaptive Biotechnologies). All the analyses (TCR richness estimation and clonality) were performed using the online tool of ImmunoSEQ Analyzer 3.0.

Mouse-Related Experiments

326 B6.129P2-Park7Gt (XE726) Byg/Mmucd mice were developed and characterised as described elsewhere (Pham et al., 2010, Genes Brain Behav 9, 305-317). The DJ-1^{-/-} (KO), DJ-1^{+/-}, DJ-1^{+/+} (WT) mice used in our experiments were gender- and age-matched siblings generated from heterozygous DJ-1^{+/-} breeding pairs. All mice were maintained in our SPF animal facility and all animal experimental procedures were performed following the approval of the Animal Welfare Society (AWS) of University of Luxembourg and Luxembourg Institute of Health.

FACS for Murine Cells

The same number of cells from spleen or peripheral lymph nodes were incubated with anti-mouse CD16/CD32 Fc blocker (553141, BD Biosciences) and then stained with various surface and intracellular antibodies. Cell numbers were determined by CASY (Innovatis AG). For cytokine staining, cells were fixed and permeabilized with Cytofix/Cytoperm buffer (554714, BD Biosciences). Anti-IL-2 (25-7021-82, eBioscience), anti-IFNγ (564336, BD Biosciences), anti-IL-5 (554395, BD Biosciences), anti-IL-13 (48-7133-82, eBioscience), anti-IL-17A (53-7177-81, eBioscience) and anti-IL-10 (505028, Biolegend) antibodies were diluted in Perm/Wash buffer (554714, BD Biosciences). Intracellular staining for Foxp3 (17-5773-82, eBioscience), CTLA4 (12-1522-83, eBioscience), Helios (137220, Biolegend) and Ki-67 (48-5698-82, eBioscience) was performed by using the Foxp3 Staining Kit (00-5523-00, eBioscience). Samples were measured on a BD LSRFortessa™ and data were analysed with FlowJo (v10, Tree Star).

Intracellular Cytokines Measurement

For intracellular cytokine measurement, cells (2E5) from spleen and draining lymph nodes were restimulated by 50 ng/ml PMA (Phorbol 12-myristate 13-acetate, P8139, Sigma-Aldrich) and 750 ng/ml ionomycin (10634, Sigma-Aldrich) in the presence of Golgiplug (555029, BD Biosciences) and Golgistop (554724, BD Biosciences) for 5 hrs in 96-well plates. Following cell surface staining, cells were fixed and permeabilized with Cytofix/Cytoperm buffer (554714, BD Biosciences).

EAE Model and Histology

The EAE model was performed as previously described (Mak et al., 2017, Immunity 46, 675-689). In brief, each mouse was injected subcutaneously (s.c.) with 115 μg MOG35-55 peptide (Washington Biotech) emulsified in CFA (263810, Difco) plus an intraperitoneal (i.p.) injection of 300 ng pertussis toxin (NC9675592, List Biological) on days 0 and 2. Clinical signs were assessed daily as described (Brustle et al., 2012, J Clin Invest 122, 4698-4709). Spinal cords and brains were collected at day 30 post EAE induction for histological analyses. Brains were fixed in 4% Paraformaldehyde for 48 hrs and stored until further processing in 0.1 M PBS. Brains were dehydrated in ascending serial dilution of ethanol/Xylol (Carl Roth, Germany) and embedded in paraffin. Brains were cut coronally in 8 μm-thick sections on a microtome (Leica Biosystems, Germany) and mounted on slides. Sections were dried overnight at 37° C. and dewaxed (2×15 min Xylol) and rehydrated in an ascending ethanol series. After washing in H2O sections were transferred to 0.01 M Na-Citrate pH 6 (Mac-3 staining) or to Target Retrieval solution (pH 9, S236884-2, Agilent DAKO; CD3 staining) for antigen retrieval (in microwave 900 W (Mac-3 staining) or 600 W (CD3 staining). Before being incubated in a blocking solution (10% FCS, 0.05% Triton X100 in PBS) in a humid chamber, sections were cooled down, washed in 0.1 M PBS-0.1% Tween-20 and endogenous peroxidase was inactivated by 0.2% H2O2/PBS. Sections were incubated with primary antibodies overnight (anti-CD3 antibody:dilution 1:100, DAKO A0452; anti-Mac-3 antibody:dilution 1:50; CD107b, BD Pharmingen). The next day slides were washed in 0.1 M PBS and incubated for 1 hr with secondary antibodies diluted 1:250 in the blocking solution (anti-CD3 staining: 111-065-003, AffiniPure Goat Anti-rabbit IgG (H+L), Jackson ImmunoResearch; anit-Mac3 staining: 112-065-003, AffiniPure Goat Anti-rat IgG (H+L), Jackson ImmunoResearch). After another wash in 0.1 M PBS sections were incubated in ABC solution (ABC Vectastain PK6100, Biozol), washed in 0.1 M Tris-HCl and HRP reaction initiated with 0.5% 3′-diamiobenzidine (D-5637-5G, Sigma-Aldrich), 0.000025% H2O2 in 0.1 M Tris-HCl. Reaction was performed in the dark and monitored regularly. Reaction was stopped by washing in 0.1 M PBS and sections were counterstained with hematoxylin (Mayer's hematoxylin, MHS1, Sigma-Aldrich) dehydrated in an ascending ethanol series and coverslipped in PERTEX mounting media (LEIC801, VWR). Microphotographs were taken on a ZEISS Axio Scope using an AxioCam Camera. Images were adjusted for brightness and contrast using Adobe Photoshop.

Seahorse Metabolic Assay

Naïve CD4 and CD8 T cells (CD62L^{high}CD44^{low}), effector memory CD4 T cells (CD62L^{low}CD44^{high}), central memory CD8 T cells (CD62L^{high}CD44^{high}) and CD4⁺CD25^{high} Treg cells from the spleen of old Dj-1 (˜45 weeks) KO and WT mice were sorted on BD FACSAria™ III. 4E5 freshly isolated cells were allowed to rest for 3 hrs in the complete RPMI media before being plated in wells by using Cell-TAK (354240, Corning). Tregs were pooled together from different mice of the same group to reach 4E5/well due to a limited number of Tregs in each individual mice. The oxygen consumption rate (OCR) was measured in XF base medium (102353-100, Agilent Technologies, from the Seahorse XF Cell Mito Stress Test Kit), containing 1 mM pyruvate (S8636, Sigma-Aldrich), 2 mM glutamine (G8540, Sigma-Aldrich) and 25 mM glucose (G8769, Sigma-Aldrich), under basal conditions and in response to 1 μM Oligomycin (103015-100, Agilent Technologies), 1.5 μM FCCP (carbonyl cyanide-4 (trifluoromethoxy) phenylhydrazone) (103015-100, Agilent Technologies) and 1 μM Rotenone and 1 μM Antimycin A (103015-100, Agilent Technologies) by the Seahorse XF96 analyser (Agilent). The results were analysed by Wave 2.6.0 (Agilent Technologies).

Adoptive Transfer of Naïve CD8 T Cells

Naïve CD8 T cells in spleens and pLNs were isolated from the young or old DJ-1^{-/-} mice and their WT littermates, respectively. Then 3×10⁵ naïve CD8 T cells as donor cells were adoptive transferred into the gender-matched Rag1^{-/-} recipient mice aged 8-12 weeks old by i.v. injection in 200 μl of PBS. 6 weeks later, the mice were sacrificed and the spleen was taken to analyse different subsets and various markers on CD8 T cells.

Statistical Analysis

P values were calculated with non-paired two-tailed Student t-test (Graphpad prism or Excel) as specified in Figure legend. The EAE clinical scores between WT and KO groups were compared by paired two-tailed Student t-test (Graphpad). All error bars represent the standard deviation. The P-values associated with Pearson correlation analysis were from a two-tailed test generated by GraphPad Prism.

### Example 1: Dj-1 Depletion Halts Immunoaging in Old Mice

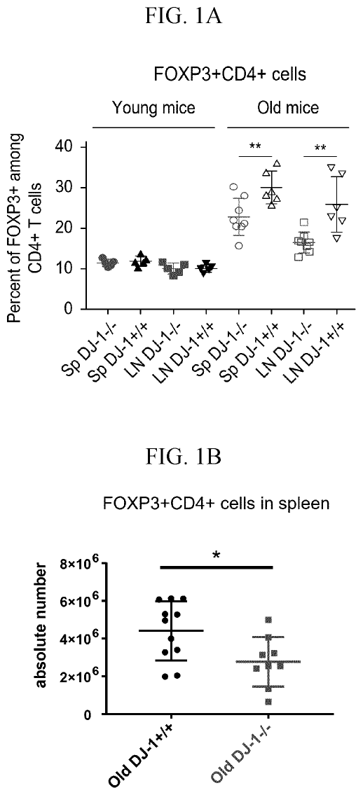

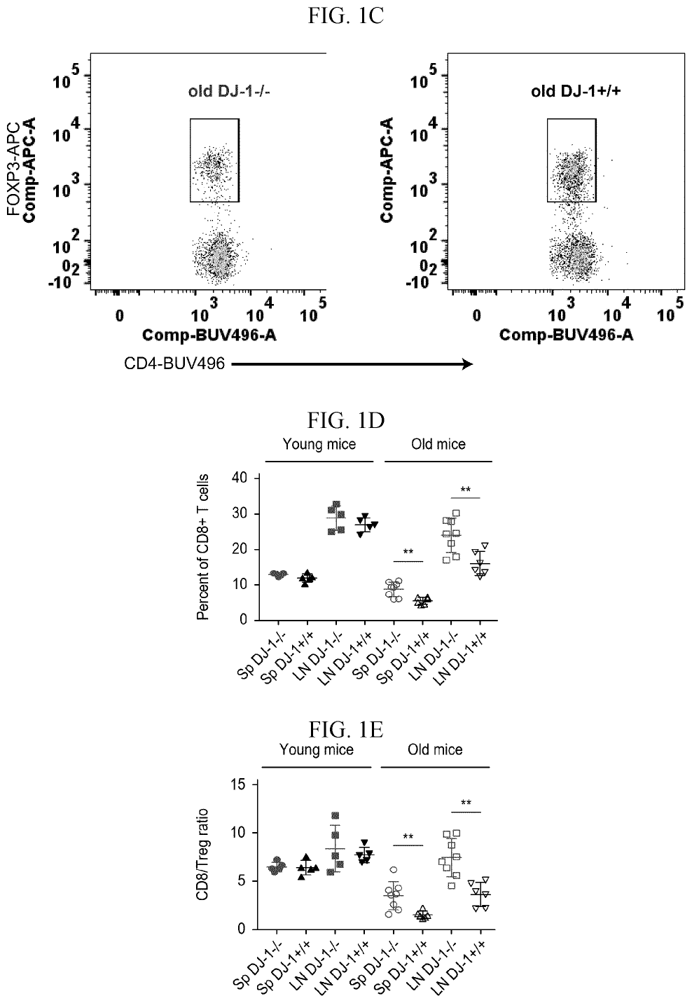

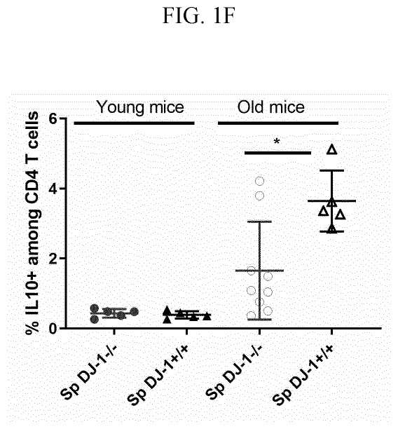

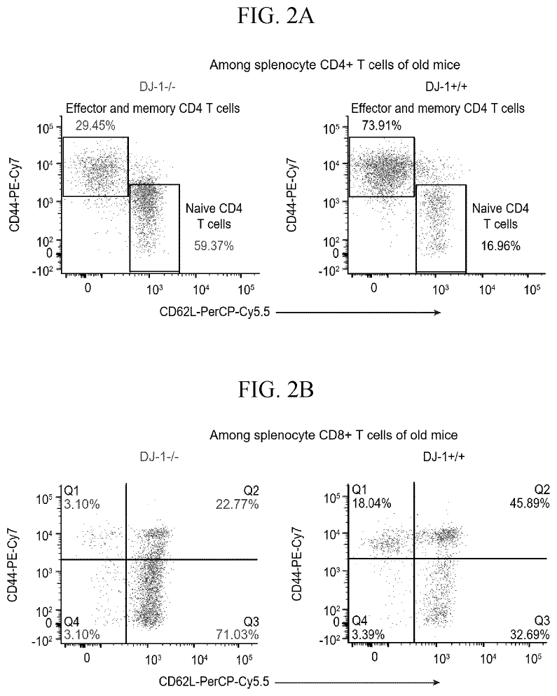

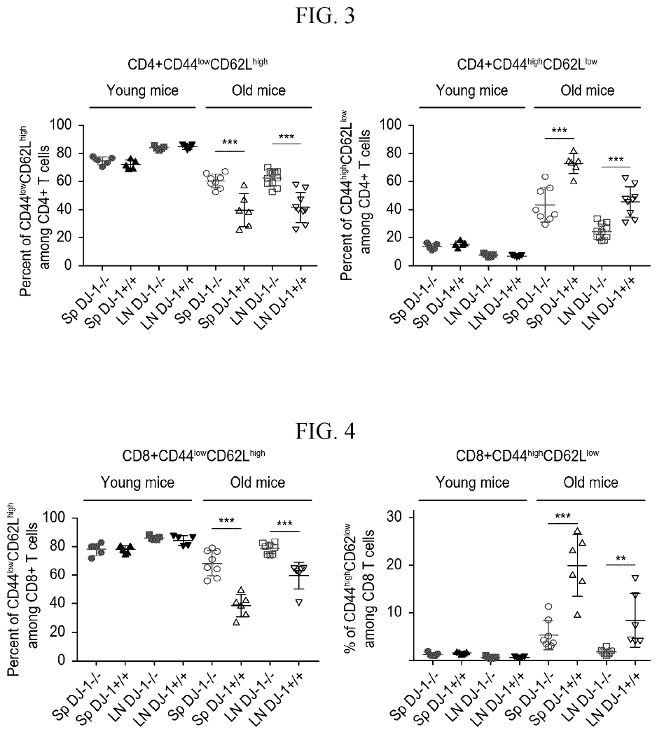

The present inventors demonstrated much lower percentages and absolute number of Tregs in the old but not in young DJ-1 KO mice relative to the age- and gender-matched littermate control WT mice (FIG. 1A, FIG. 1B and FIG. 1C). The inventors subsequently set out to evaluate whether there was any significant change related to immunoaging, as Tregs are essential for the formation and maturation of CD8 memory T. Therefore, various relevant aspects manifested during immunoaging, such as the exhaustion and activation markers of CD4, CD4⁺FOXP3⁺ Tregs, and CD8 T cells as well as various memory and naïve subsets, were investigated. Although the frequency of total CD4 T cells was not significantly changed, there was a significant increase in the percentage of total CD8 T cells in pLNs (peripheral lymph nodes) and the spleen of old DJ-1 KO mice (FIG. 1D). Both the frequency and total number of Tregs were significantly decreased in old DJ-1 KO mice (FIG. 1A, FIG. 1B, FIG. 1C), which restricted the formation of CD8 memory T cells, although the individual TSF was significantly augmented. The ratio between CD8 T cells and Tregs was decreased in old but not young DJ-1 KO mice (FIG. 1E). Consistent with reduced Tregs, the frequency of splenic IL-10-producing CD4+ cells following in vitro PMA/ionomycin stimulation was significantly decreased in old DJ-1 KO mice vs. WT littermates. Notably, accumulation of CD4 and CD8 T CD44^{high}CD62L^{low} effector memory (Tem) cells was sharply reduced, while the naïve CD44^{low}CD62L^{high} T cells were significantly augmented in spleen (Sp), peripheral blood and pLNs of old Dj-1 KO mice as compared to age-matched DJ-1^{+/+} WT mice (FIG. 2A, FIG. 3). The frequency of CD8 central memory T cells (Tcm) was also dramatically reduced in old Dj-1 KO mice as compared to age-matched DJ-1^{+/+} WT mice (FIG. 2B, FIG. 4).

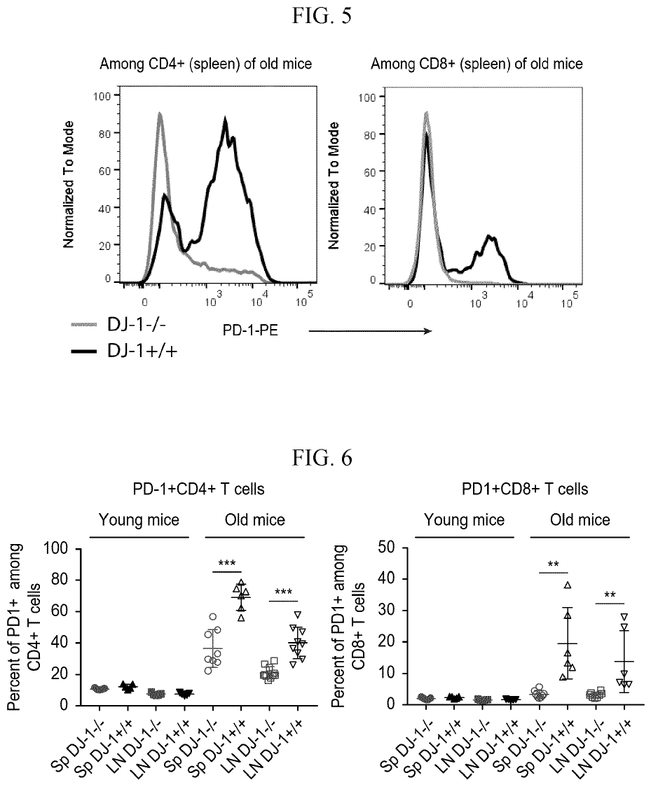

Interestingly, the frequency of positive cells for all of the critical proliferation, exhaustion and activation markers, such as Ki67, PD-1, CTLA4, CD69, ICOS and Helios was significantly reduced among total CD4 and CD8 T cells in spleens and/or lymph nodes of old Dj-1 KO mice relative to those of the WT controls (FIG. 5, FIG. 6, results shown for PD-1). The decrease in these markers was essentially due to the reduced memory T cell compartment since these markers are predominantly expressed in memory T cells vs. Tn cells. The selective effect on immune status of old but not young mice was not directly attributable to a possible expression change of Dj-1 with age in WT mice since there was no significant difference in Dj-1 expression between two age groups in various T-cell subsets. Together, these data demonstrated a juvenile and less-exhausted phenotype of CD4 and CD8 T cells in old Dj-1 KO mice compared with that in age-matched WT littermates.

### Example 2: Dj-1 Deficiency Enhances Antigen-Specific Response in Old but not Young Mice

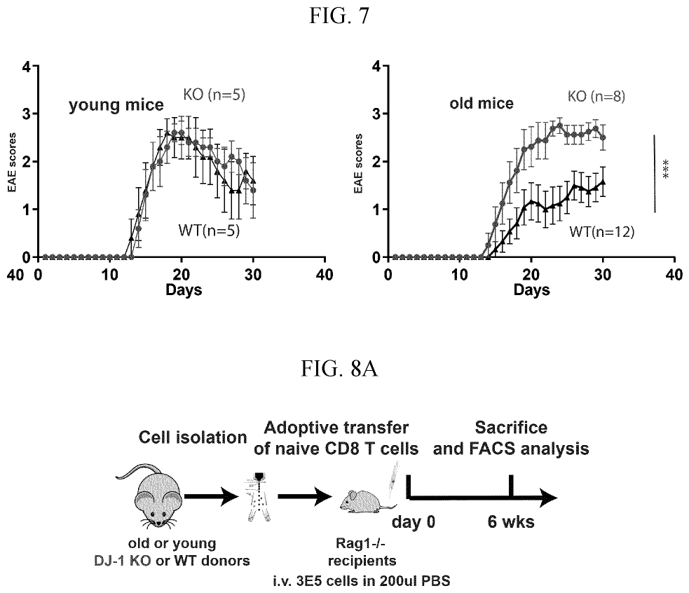

To examine whether the DJ-1-depletion-driven accumulation of Tn and reduced Tregs contributed to the physiopathogenesis in vivo, we used the myelin oligodendrocyte glycoprotein (MOG₃₅₋₅₅)-induced experimental autoimmune encephalomyelitis (EAE) model to induce antigen-specific T-cell activation. Interestingly, in line with the aging-dependent shift in homeostatic immune status, only old Dj-1 KO mice, but not young Dj-1 KO mice, exhibited a deteriorated EAE symptoms relative to their WT littermates (FIG. 7). The worsened symptoms might also be attributable to diminished PD-1 expression in T cells (FIG. 5, FIG. 6) since blockade of PD-1 results in accelerated and more severe EAE disease. Furthermore, we observed a higher secretion of key EAE-relevant cytokines, such as the Th1-cell-derived cytokine GM-CSF as well as Th17 cytokines IL-17A and IL-17F, although to a lesser extent, by MOG-restimulated splenocytes isolated from old Dj-1 KO mice relative to those from WT mice.

Following EAE induction, the frequency of circulating naïve CD4 and CD8 T cells was still higher in old Dj-1 KO mice relative to those in WT mice at day 30. Accordingly, the fractions of circulating memory CD4 and CD8 T cells were lower. However, the frequency of memory T cells in draining lymph nodes (dLNs), which were decreased in homeostatic old KO mice (FIG. 3, FIG. 4), turned out to be similar between KO and WT mice due to increased MOG-specific responses and accordingly enhanced memory cell accumulation in the old KO mice. These results together demonstrate that the accumulation of Tn and reduced Tregs enhanced antigen-specific responses in elderly Dj-1 KO mice.

### Example 3: DJ-1 Depletion Intrinsically Attenuates the Development of Senescent CD8 T Cells

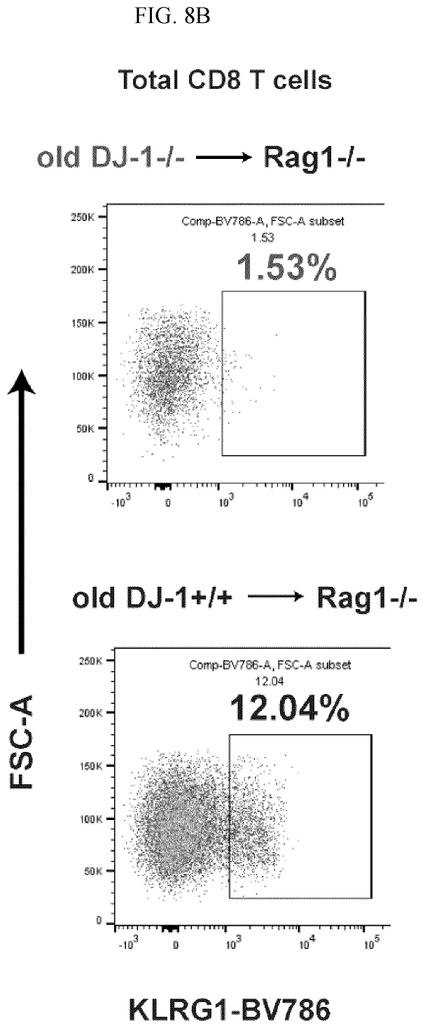

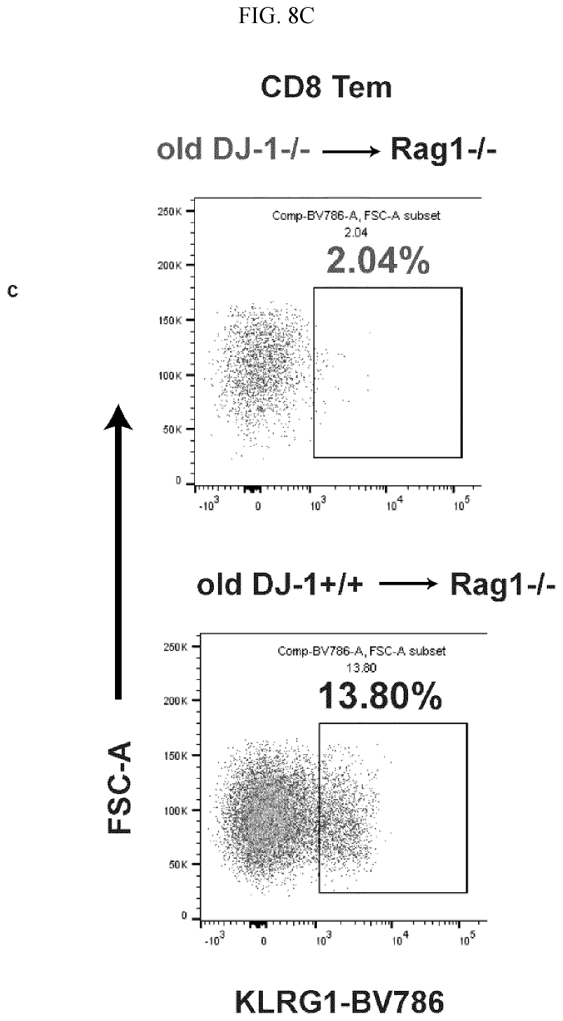

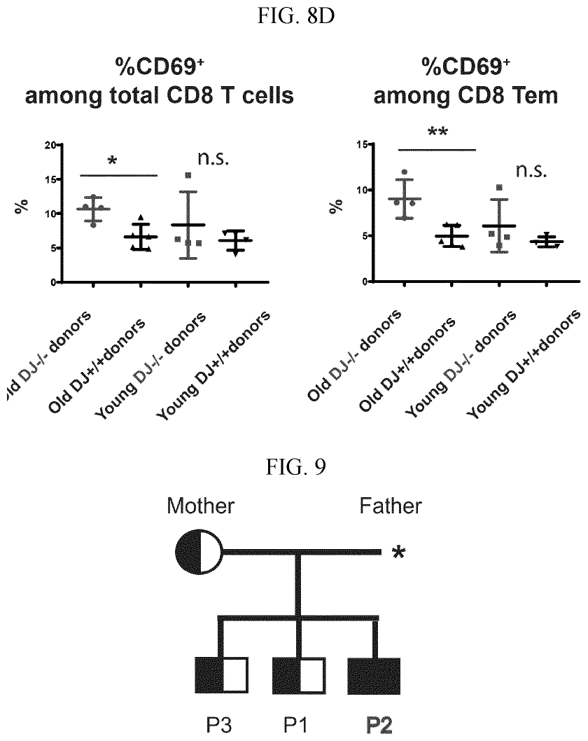

To check whether DJ-1 could regulate memory and senescence development, we transferred the naïve CD8 T donor cells from old or young DJ-1 KO or WT littermates into the young Rag1^{-/-} recipient mice (FIG. 8A). Rag1^{-/-} mice lack mature T and B cells, therefore providing an empty space for the repopulation/reconstitution of donor cells. To monitor the senescence development, the expression of a well-accepted senescence markers was evaluated in murine T cells, i.e., KLRG1. There were lower percentage of KLRG1⁺ CD8 T cells (FIG. 8B) and KLRG1⁺ EM CD8 T cells (FIG. 8C) developed from the old DJ-1 KO naïve CD8 T donor cells compared to the old WT donor cells. No significant difference of KLRG1 expression was observed between the young Dj-1 KO and WT donor cells (results not shown). Moreover, there were higher percentage of CD69⁺ CD8 T (FIG. 8D, left) and CD69⁺ EM CD8 T cells (FIG. 8D, right) from the old DJ-1 KO donor cells compared to that from the old WT donor cells, which is, however, not the case for that from young donor cells (FIG. 8D).

### Example 4: DJ-1 Ablation Confers PD Patients a Juvenile Immune System

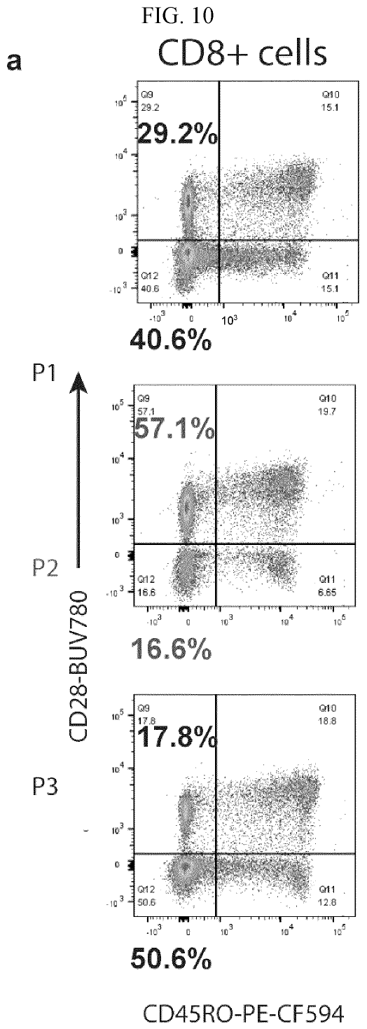

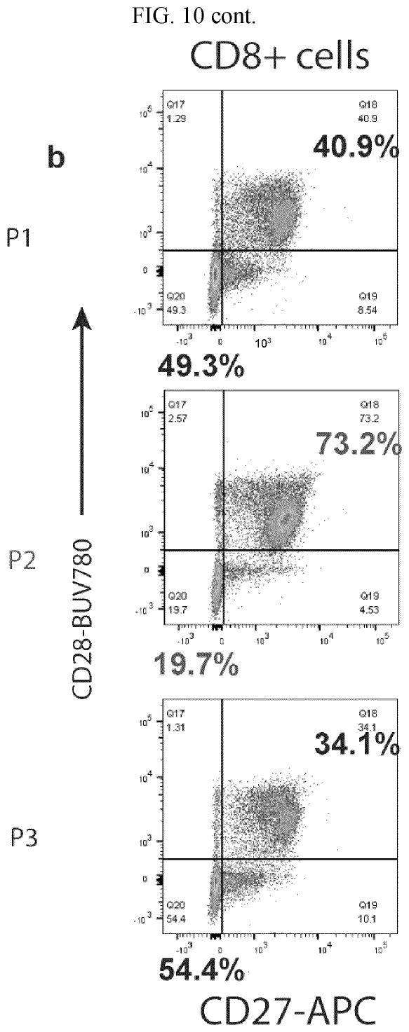

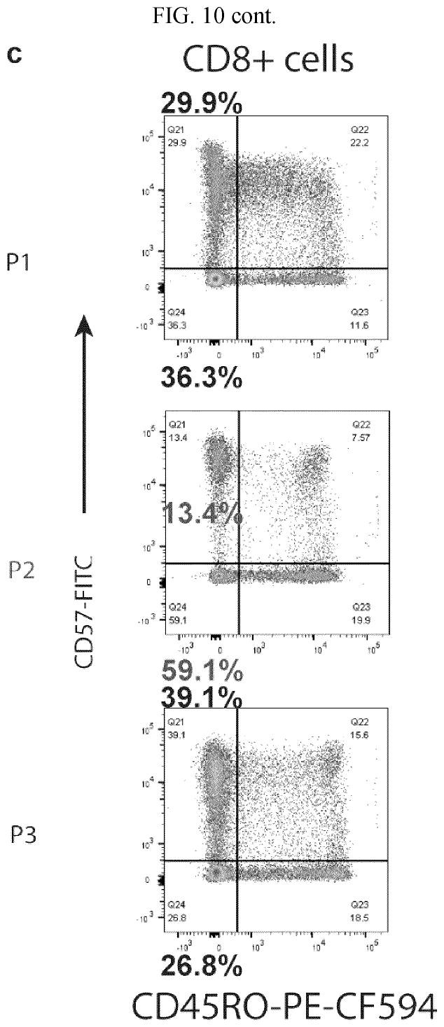

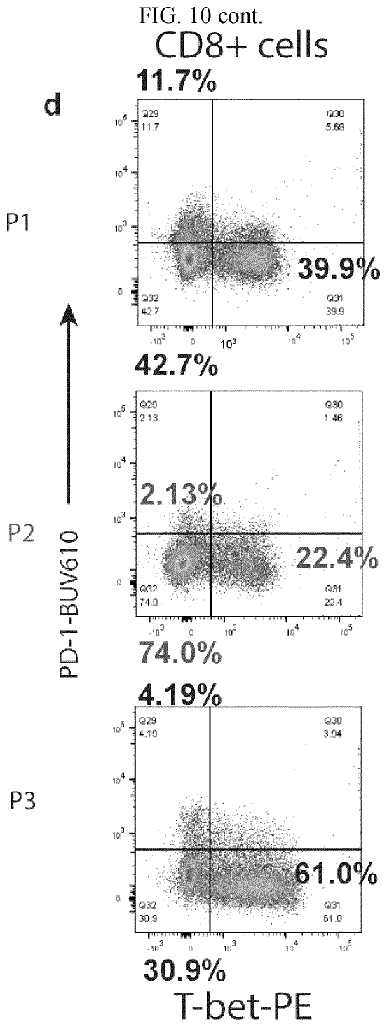

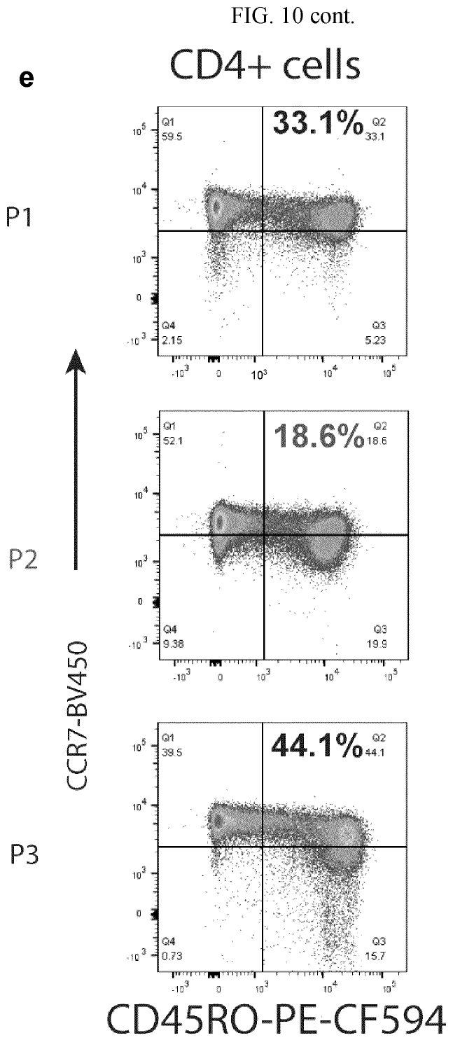

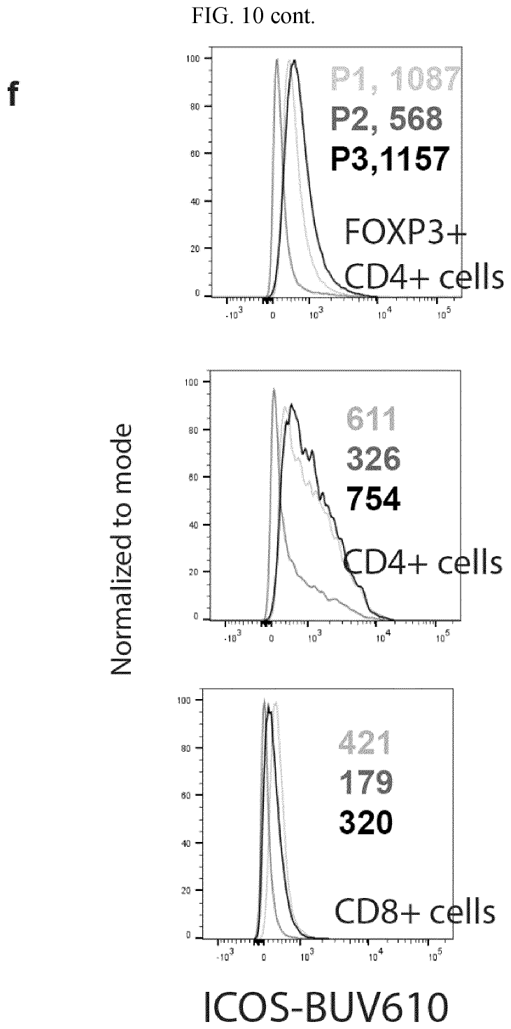

To confirm whether our observation in KO mice had clinical relevance, we analysed the activation, exhaustion and senescence markers of T cells in one PD patient carrying the homozygous c.192G>C mutation in the DJ-1 gene and two siblings, who are unaffected heterozygous carriers of the same PD causing mutation. In comparison with the unaffected age-matched siblings (FIG. 9, 56-63-year old), the patient without DJ-1 expression had an approximately twofold higher frequency of naïve non-senescent CD8 T cells, such as CD45RO⁻CD28⁺, CD45RO⁻CD27⁺, CD45RO⁻CD57⁻ and CD27⁺CD28⁺ CD8 T cells (FIG. 10A-C). This relationship also held true for CD4 T cells, although to a lesser extent (FIG. 10E). The frequency of non-exhausted CD8 T cells, defined as PD-1⁻Tbet⁻ and PD-1⁻Eomes⁻, were also nearly doubled in the patient's blood (FIG. 10D). PD-1-expressing CD4 T cells were also decreased, which was mainly due to reduced accumulation of CD45RO⁺ T cells, as PD-1 is mainly expressed in memory T cells. Again, consistent with the elderly mice data, the activation degree of CD4 and CD8 T (ICOS+) cells was decreased (FIG. 10F) in the patient without DJ-1 expression. The ICOS+ T cells were predominantly CD45RO⁺ T cells, especially among Tregs or CD4 T cells. Again, similar to the observation in aged murine T cells, the frequency of FOXP3⁺CD4⁺ Tregs was decreased in the affected patient (FIG. 10F). These data are therefore highly consistent with the murine results and demonstrate that DJ-1 plays an important and conserved role in promoting T-cell aging across different species.

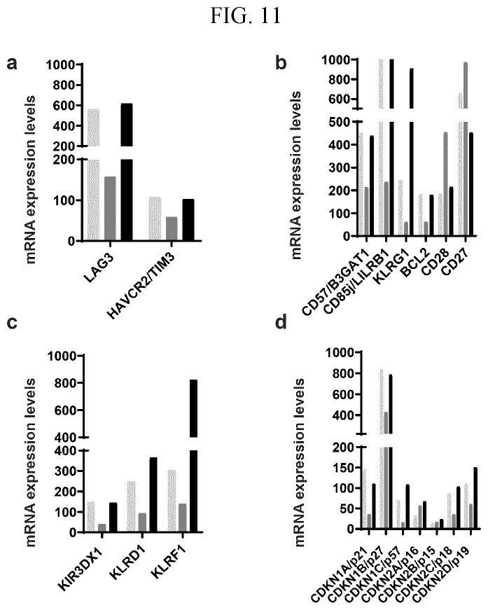

To obtain more comprehensive understanding of the change of transcriptomic profiling caused by loss of DJ-1, a microarray analysis of CD8 T cells isolated from the peripheral blood of the three participants was performed. Accordant with our FACS analysis outcome, we observed the mRNA expression of some exhaustion genes, such as LAG3 and TIM3, were strikingly downregulated in DJ-1 deficient CD8 T cells (FIG. 11A). Moreover, the mRNA expression levels of some T cell senescence related genes, such as CD57, CD85j and KLRG1 were much lower in CD8 T cells of DJ-1 homozygous mutated patient (FIG. 11B). The expression of costimulatory genes CD28 and CD27 were much higher in the DJ-1 deficient CD8 T cells (FIG. 11B), which is consistent and complimentary to the phenotypes which were observed before.

One NK cell related receptor KIR3DX1 and two lectin-like receptors KLRD1/CD94 and KLRF1/NKP80 were highly expressed in the CD8 T cells of two healthy controls. However, the expression levels of these three NK cell-related markers were much lower in the DJ-1 homozygous mutated CD8 T cells (FIG. 11C).

Another major feature of senescent T cells is proliferative arrest. The involvement of cyclin-dependent kinase inhibitors (CKI) is crucial for the regulation of cell cycle arrest of senescent cells. Several of CKI genes have been reported to be upregulated during the aging. Therefore, two major families of CKI, the Cip/Kip family including CDKN1A/p21, CDKN1B/p27, CDKN1C/p57, and the INK4 family including CDKN2A/p16, CDKN2B/p15, CDKN2C/p18 and CDKN2D/p19 were investigated. p21 mRNA expression in DJ-1 homozygous mutated human CD8 T cells was much lower in contrast to the control CD8 T cells (FIG. 11D), which further demonstrated that loss of DJ-1 caused a senescent phenotype of CD8 T cells. No significant difference of p16 and p15 expression was observed in CD8 T cells among these three participants (FIG. 11D). Moreover, the mRNA expression levels of p27, p57, p18 and p19 were all much lower in DJ-1 homozygous mutated human CD8 T cells compared with the DJ-1 heterozygous mutated CD8 T cells (FIG. 11D). Taken together, DJ-1 modulates T cell cycle progression by affecting the expression of key CKIs.

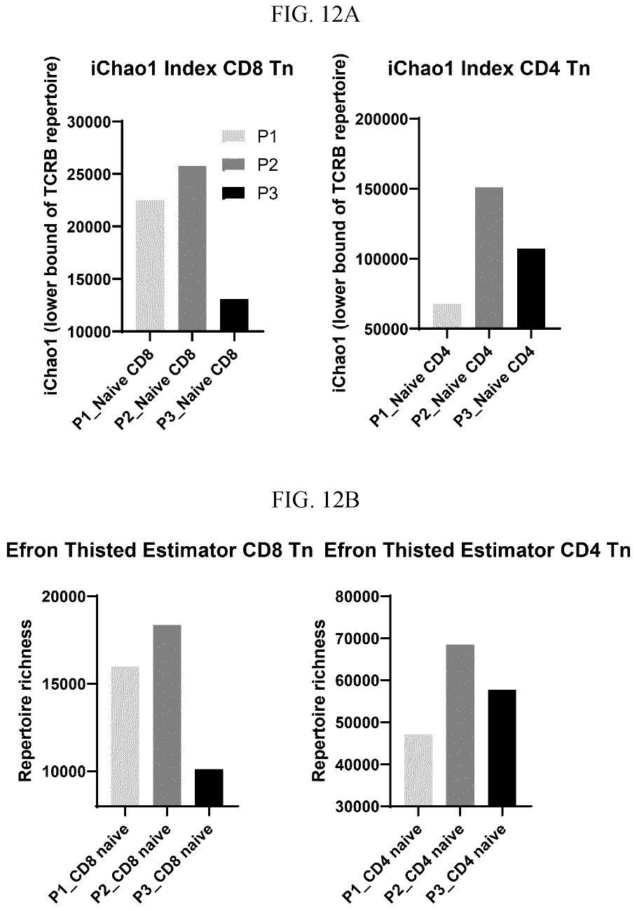

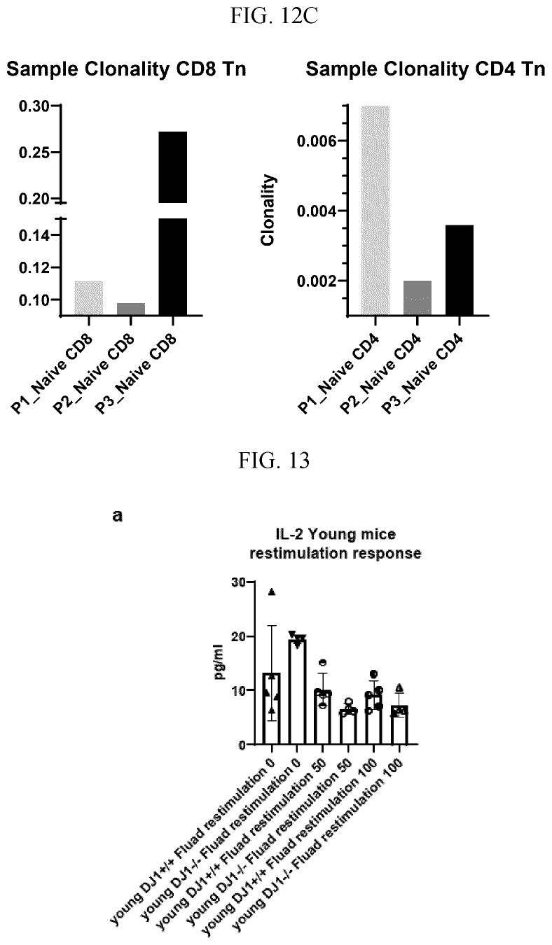

Another critical hallmark of immunoaging is the reduced diversity of TCR repertoire in naïve T cells. To examine whether the reduced immunoaging in cellular levels is also reflected in TCR repertoire, we sequenced the TCR beta repertoire of sorted naïve CD4 and CD8 T cells employing the high-throughput TCR sequencing platform provided by Adaptive Biotechnologies. Diversity has two independent components, clonality and richness. Using the manufacture online analysis tool ImmunoSEQ 3.0, we first estimated the TCR repertoire richness using two well-accepted non-parametric estimating methods (iChao 1 estimator and Efron-Thisted estimator). Although both richness estimation methods often underestimate the richness for a smaller small size (which is the case for the number of sequenced cells from the patient with DJ-1 loss-of-function deficiency, i.e., P2), both estimators still predicted a higher TCR beta richness for the patient with DJ-1 deficiency than that of the healthy siblings (FIG. 12A and FIG. 12B). A lower clonality score (1-normalized Shannon's Entropy) indicated there was fewer clonal expansion. The clonality was also lower in the patient with DJ-1 deficiency for both naïve CD8 (FIG. 12C, left) and CD4 T cells (FIG. 12C, right), especially apparent for CD4 naïve T cells. Therefore, the TCR repertoire diversity within naïve T cells was also reduced in the patient with DJ-1 deficiency.

Conclusion

The results in both a PD patient (Example 4) and DJ-1 KO mice (Examples 1-3) identified an unanticipated critical causative link between DJ-1 and immunoaging. The data illustrate that DJ-1 binds to the essential entry point of the TCA cycle, PDHB, preferentially in Tregs relative to Teffs (mechanistic data not shown here), thus promoting Treg-driven anti-inflammatory responses and providing a direct molecular link between metabolism and Treg-mediated immune responses.

In contrast to the natural aging process, wherein the frequency of Tregs increases, while the frequency of naïve T cells decreases with age, the present inventors identified a reduction in the Treg frequency of old DJ-1 knockout (KO) mice relative to that of the age- and gender-matched wildtype (WT) mice. Meanwhile, a significant increase of naïve T cells (Tn) and a significant decrease in the compartment of memory T cells was observed in old DJ-1 KO mice relative to that of WT mice. During the natural aging process, the exhaustion markers, such as PD-1 increase on T cells. On the contrary, this invention identified the loss of DJ-1 caused a reduction of the exhausted CD4 and CD8 T cells. The mice data demonstrated that DJ-1 depletion reduced immunoaging.

In line with mice data, the inventors have also observed a younger immune system when they analysed the blood from a patient with DJ-1 deficiency relative to the age- and gender-matched healthy siblings with DJ-1 heterozygous mutation. More specifically, in the patient with DJ-1 deficiency vs. the siblings, reduced exhaustion and senescence in T cells were observed accompanied with an enhanced TCR repertoire diversity, which is supposed to be decreased during natural aging. In short, the inventors have demonstrated that DJ-1 depletion plays an unexpected and conservative pivotal role in slowing down immunoaging.

### Example 5: Use of DJ-1 Inhibitors to Treat Vaccination Inefficiency in a Subject According to Embodiments of the Present Invention

Several DJ-1 inhibitors, in particular small molecule DJ-1 inhibitors, are provided. Following DJ-1 inhibition by a DJ-1 inhibitor, in particular a small molecule DJ-1 inhibitor, the DJ-1 activity is tested in vitro in different types of isolated primary immune cells.

After in vitro testing, one of the DJ-1 pharmaceutical inhibitors is administered in mice for a period, e.g. between 1-3 months, with an optimal dose (nM or mM range). The potential alterations in peripheral immunological phenotypes and statuses are examined. The interval of administration is dependent on the drug half-life, i.e., the pharmaceutic kinetics (known as PK) of the inhibitor. The administration mode is oral delivery or injection.

The evolution of the frequency and absolute number of CD4⁺FOXP3⁺ Tregs (or CD4⁺CD25⁺ T cells) among total CD4 T cells is tested in blood following DJ-1 inhibition by administration of the DJ-1 inhibitor, in particular the small molecule DJ-1 inhibitor. The evolution of the frequency and absolute number of naïve/memory CD4 and CD8 T cells is tested in blood following DJ-1 inhibition by administration of the DJ-1 inhibitor, in particular the small molecule DJ-1 inhibitor.

The dynamic change of expression levels of several relevant molecular and functional markers, such as exhaustion and senescence markers of different immune subsets is tested in blood following inhibition of DJ-1 by the DJ-1 inhibitor, in particular the small molecule DJ-1 inhibitor. The corresponding parameters in the spleen are tested when sacrificing the mice after treatment by one of the small molecule DJ-1 inhibitors or are tested in humans such as elderly humans.

If successful, an enhanced percentage of antigen-specific cells among total CD4 or CD8 T cells is observed, accompanied with reduced frequency of exhaustion and senescence markers among CD4 and/or CD8 T cells. If a younger immune system is observed, follow-up influenza vaccination experiments are performed to test the physiological responses following immune challenges in WT and DJ-1 KO mice. This is done both in young and elderly mice. The antigen-specific response is analyzed using MHC-I tetramer-labelled technique to detect the frequency of antigen-specific responses.

### Example 6: Aged DJ-1^{-/-} Mice Show a Stronger Vaccination Response Compared to the DJ-1^{+/+} Littermate Controls

Materials and Methods:

Young or aged DJ-1^{+/+} or DJ-1^{-/-} mice were injected with 100 μl of seasonal influenza vaccine FLUAD (Novartis) subcutaneously twice with an interval of 3 weeks. The mice were sacrificed 9 days after the second vaccination. The 1 million splenocytes were seeded in 96-well plates followed by restimulation with different amounts of vaccine (0 or 4 or 8 μl of vaccine) in a total volume of 200 μl culture media for 3 days. The IL2 or IFNγ levels in the supernatant were measured using BD CBA kits (IL2, cat. nr. 558297; IFNg, cat. nr. 558296) following the manufacture recommendations.

Results

Following in vivo vaccination, one of the key readouts monitoring vaccination responses is the so-called in vitro vaccine restimulation responses. A higher production of vaccine related cytokines, such as IL2 or IFNγ indicates a higher vaccination response.

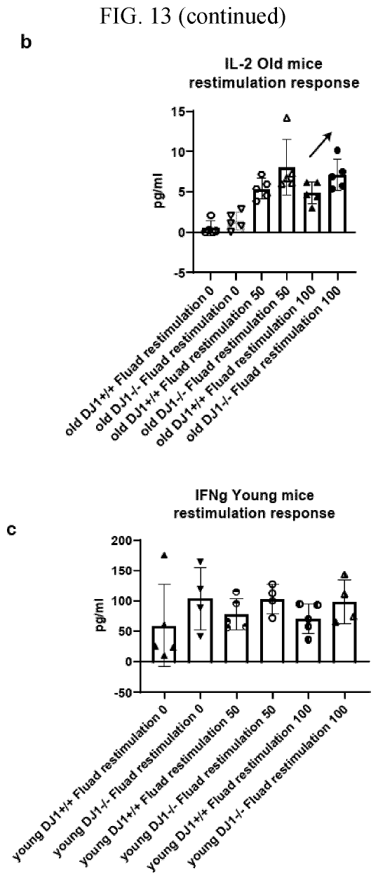

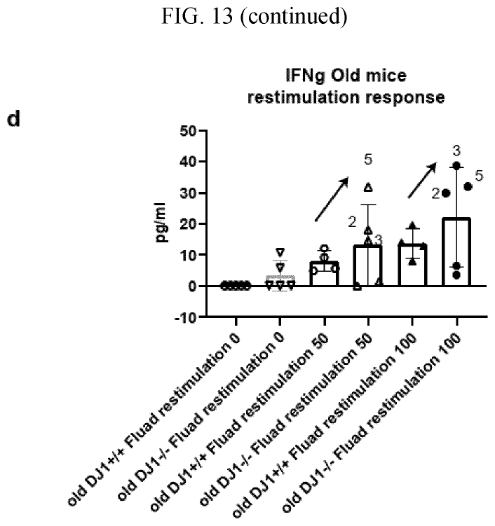

As shown in our FIG. 13, a relatively higher level of IL2 or IFNγ was observed in young DJ-1^{+/+} mice compared to aged DJ-1^{+/+} mice, which is expected as young adult mice generate stronger vaccine response than the aged counterparts. Present inventors could not observe any clear trend in the cytokine production levels between the young DJ-1^{+/+} and DJ-1^{-/-} mice following restimulation. On the other hand, vaccination response cytokine IL2 increased in old DJ-1^{-/-} mice compared to all the age- and gender-matched DJ-1^{+/+} littermate controls in the 1:100 dilution and the key vaccination response cytokine IFNγ was almost doubled in the supernatants of the restimulated splenocytes in 3 out of 5 old DJ-1^{-/-} mice compared to all the age- and gender-matched DJ-1^{+/+} littermate controls (in both 1:50 and 1:100 dilutions). Interestingly, for IFNγ, it were the same aged DJ-1^{-/-} mice that responded much higher in both vaccine restimulation dilutions. In short, a relatively stronger vaccination response, as reflected by a higher level of IL2 and IFNγ production, was observed in the aged DJ-1^{-/-} mice compared to the DJ-1⁺⁺ littermate controls.

1-17. (canceled)

18. A method for the treatment and prevention of immunoaging or an immunoaging-related disease in a subject, said method comprising administering to the subject a therapeutically or prophylactically effective amount of an inhibitor of DJ-1 (PARK7).

19. The method according to claim 18, wherein the subject has been selected to have or has a premature aging disease;

20. The method according to claim 18, wherein the subject is an elderly subject, preferably wherein the subject has an age of at least 60 years, more preferably of at least 65 years.

21. The method according to claim 18, wherein the inhibitor of DJ-1 binds to DJ-1 or a polynucleotide encoding DJ-1.

22. The method according to claim 18, wherein the inhibitor is one or more agents selected from the group consisting of a chemical substance, an antibody, an antibody fragment, an antibody-like protein scaffold, a protein or polypeptide, a peptide, a peptidomimetic, an aptamer, a photoaptamer, a spiegelmer, a nucleic acid, a gene-editing system, an antisense agent, an RNAi agent, and a soluble receptor.

23. The method according to claim 22, wherein the chemical substance is an organic molecule, preferably a small organic molecule, or wherein the nucleic acid is an oligonucleotide.

24. The method according to claim 18, wherein the inhibitor is one or more agents selected from the group consisting of an antibody, an antibody fragment, an antibody-like protein scaffold, a nucleic acid, a gene-editing system, an antisense agent, and an RNAi agent.

25. The method according to claim 24, wherein the nucleic acid or RNAi agent has a sequence identity of at least 50%, 60%, 70%, 80%, 90%, 95%, 98%, 99% or 100% to SEQ ID NO: 4.

26. The method according to claim 24, wherein the nucleic acid or the RNAi agent is provided within a plasmid vector and/or wherein the nucleic acid or the RNAi agent is modified or encapsulated by synthetic or natural nanoparticles; preferably wherein the nanoparticle is a liposomal nanoparticle.

27. The method according to claim 18, wherein the immunoaging-related disease is cancer or an infectious disease.

28. The method according to claim 19, wherein the premature aging disease is selected from the group consisting of Hutchinson-Gilford progeria syndrome (HGPS), Bloom syndrome (BS), Cockayne syndrome (CS), Mandibuloacral Dysplasia with Type A Lipodystrophy (MADA); Werner syndrome (WS), Rothmund-Thomson syndrome (RTS), Seip syndrome, xeroderma pigmentosum (XP), trichothiodystrophy (TTD), combined xeroderma pigmentosum-Cockayne syndrome (XP-CS), and restrictive dermopathy (RD); preferably wherein the premature aging disease is Hutchinson-Gilford progeria syndrome; and/or wherein the subject has been selected to have or has a premature aging disease.

29. The method according to claim 18, wherein said treatment or prevention further comprises administering to said patient a compound or composition capable of inducing an immune response

30. A method of vaccinating a subject, said method comprising, administering to said patient a composition comprising an immunogenic composition and an inhibitor of DJ-1.

31. The method according to claim 30, wherein the subject is an elderly subject, preferably wherein the subject has an age of at least 60 years, more preferably of at least 65 years.

32. The method according to claim 30, wherein the subject has a premature aging disease;

33. The method according to claim 30, wherein the inhibitor of DJ-1 is administered as an adjuvant.

34. The method according to claim 30, wherein the inhibitor of DJ-1 enhances the immune response in said subject.

35. The method of claim 30, wherein said an immunogenic composition is a vaccine and said method is a method for treating or preventing vaccination inefficiency in a subject.

36. An immunogenic composition or kit of parts comprising an inhibitor of DJ-1 and a compound or composition capable of inducing an immune response.

37. The immunogenic composition according to claim 35 wherein the immunogenic composition is a cancer vaccine.


SEQUENCE LISTING (10 sequences)

SEQ ID NO: 1 - DNA, 1127 bp, Homo sapiens
gcgttcattttcagcctggtgtggggtgagtggtacccaacgggccggggcgccgcgtccgcaggaagaggcgcggggtgcaggcttgtaaacatataacataaaaatggcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactagagcagcgaactgcgacgatcacttagagaaacaggccgttaggaatccattctcactgtgttcgctctaaacaaaacagtggtaggttaatgtgttcagaagtcgctgtccttactacttttgcggaagtatggaagtcacaactacacagagatttctcagcctacaaattgtgtctatacatttctaagccttgtttgcagaataaacagggcatttagcaaactactgattgtttcttgttttgtctctcatttcttttgtgaaattaaattccgtatcaccttcatttgcagctcttaactgtccatatggcactgaaataaaagaacagtgaccacattttacacagcaaggaggaaaggcatacaaacagaatttaagaggcttgtgattttctctgcttattagctgtgtgtttttaatgtgctattaaaaaataccaatgagg

SEQ ID NO: 2 - DNA, 921 bp, Homo sapiens
tgagtctgcgcagtgtggggctgagggaggccggacggcgcgcgtgcgtgctggcgtgcgttcattttcagcctggtgtggggcttgtaaacatataacataaaaatggcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactagagcagcgaactgcgacgatcacttagagaaacaggccgttaggaatccattctcactgtgttcgctctaaacaaaacagtggtaggttaatgtgttcagaagtcgctgtccttactacttttgcggaagtatggaagtcacaactacacagagatttctcagcctacaaattgtgtctatacatttctaagccttgtttgcagaataaacagggcatttagcaaactaaaaaaaaaaaaaaaaaaa

SEQ ID NO: 3 - protein, 189 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 4 - DNA, 21 bp, Artificial Sequence
  anti-DJ-1 siRNA
aatggaggtcattacacctac

SEQ ID NO: 5 - DNA, 20 bp, Homo sapiens
gtcacaaacagtgcacctac

SEQ ID NO: 6 - DNA, 20 bp, Homo sapiens
atggttgctgtctcatcagc

SEQ ID NO: 7 - DNA, 24 bp, Homo sapiens
gatggggaaactgaggcttaggaa

SEQ ID NO: 8 - DNA, 24 bp, Homo sapiens
acccccaatctcaccccacaaata

SEQ ID NO: 9 - DNA, 24 bp, Homo sapiens
ctcgctccaggaccttcttcacaa

SEQ ID NO: 10 - DNA, 24 bp, Homo sapiens
gcttcctgctcctcaaaactaaca